(19) United States
(12) Patent Application Publication
(10) Pub. No.: US 2012/0101508 A1
(43) Pub. Date: Apr. 26, 2012

(54) METHOD AND DEVICE FOR CONTROLLING/COMPENSATING MOVEMENT OF SURGICAL ROBOT

(76) Inventors: Seung Wook CHOI, Seongnam-si (KR); Min Kyu Lee, Yongin-si (KR); Dong Myung Min, Hwaseong-si (KR)

(21) Appl. No.: 13/276,354

(22) Filed: Oct. 19, 2011

(30) Foreign Application Priority Data
Oct. 21, 2010 (KR) 10-2010-0102917

(51) Int. Cl. A61B 19/00; G05B 19/02

(52) U.S. Cl. 606/130; 700/259; 901/2; 901/47

(57) ABSTRACT

A movement compensating device of a surgical robot in which a surgical operation processing unit mounted with a surgical instrument is coupled to one end of a body section includes: an image information creating unit that creates image information corresponding to an image signal supplied from a camera unit; a recognition point information analyzing unit that creates analysis information on a distance and an angle between a recognition point recognized from image information pieces corresponding to a predetermined number of image frames and a predetermined reference point; a variation analyzing unit that creates variation information in distance and angle between two analysis information pieces continuously created; and a control command creating and outputting unit that creates and outputs a control command for adjusting the position of the surgical operation processing unit so that the variation in distance and angle included in the variation information be 0 (zero).

### CROSS-REFERENCE TO RELATED APPLICATIONS

This application claims the benefit of Korean Patent Application No. 10-2010-0102917 filed with the Korean Intellectual Property Office on Oct. 21, 2010, the disclosures of which are incorporated herein by reference in their entirety.

### BACKGROUND

The present invention relates to movement controlling/compensating method and device of a surgical robot.

A medical operation means an act of cutting, incising, or treating skins, mucous membranes, or other tissues to cure diseases by the use of mechanical instruments. Particularly, an abdominal operation or the like of incising and opening the skin of an operation site and treating, shaping, or removing organisms therein accompanies side effects such as bleeding, patients' pain, scars and thus an operation using a robot has attracted attention as an alternative.

A surgical robot system generally includes a master robot and a slave robot. The master robot and the slave robot may be embodied independently or may be incorporated into a body.

When an operator manipulates a control device (such as a handle) disposed in a master robot, surgical instruments coupled to a robot arm of a slave robot or grasped by the robot arm are operated to perform a surgical operation.

The instruments are inserted into a human body via a medical trocar. The medical trocar is a medical instrument typically used to approach the abdominal cavity. A laparoscope, an endoscope, and the like are inserted into a human body via the medical trocar.

In the surgical robot system according to the related art, when it is intended to shift the position of a slave robot in the state where a surgical instrument or the like is inserted into a human body via a medical trocar for a surgical operation, the surgical instrument or the like should be drawn out of the human body, the position of the slave robot should be shifted, and then the surgical instrument or the like should be inserted into the human body via the medical trocar to restart the surgical operation.

When the slave robot is made to move in the state where the surgical instrument or the like is inserted into the human body via the medical trocar, the surgical instrument or the like is made to together move along the movement trace of the slave robot, thereby causing a severe problem for a patient into which the surgical instrument or the like is inserted.

However, the process of drawing out the surgical instrument or the like from the human body for the purpose of the movement of the slave robot, allowing the slave robot to move, and then inserting the surgical instrument or the like into the human body again requires much time to cause the elongation of the operation time, whereby a severe feeling of fatigue is given to a doctor who is performing the surgical operation with a high tension.

Therefore, there is a need for development of a surgical robot system in which a surgical robot can freely move during a surgical operation. In the surgical robot system according to the related art, in order to slightly shift a surgical robot body in the state where the surgical robot including the surgical robot body (lower body) mounted with a movement unit and a part (upper body) mounted with robot arms is docked, the course of undocking the surgical instruments mounted on the robot arms, causing the surgical robot body to move, and then docking the surgical instruments with the robot arms again should be undergone. However, when the upper body mounted with the robot arms can rotate and move with the movement of the surgical robot body (the lower body), it is possible to save or skip the undocking and re-docking processes.

The surgical robot system according to the related art has a problem that an operator or an operation assistant should manually cause a slave robot to move.

The above-mentioned related art is technical information possessed to make the invention or learned in the course of making the invention by the inventor, and cannot thus be said to be technical information known to the public before filing the invention.

### SUMMARY

An advantage of some aspects of the invention is that it provides movement controlling/compensating method and device of a surgical robot, which can cause the surgical robot to move to a desired position in a state where a surgical instrument and the like are inserted into a human body.

Another advantage of some aspects of the invention is that it provides movement controlling/compensating method and device of a surgical robot, which can allow the surgical robot to freely move to a desired position in response to an operator's control command when it is intended to cause the surgical robot to move during the surgical operation on a patient.

Another advantage of some aspects of the invention is that it provides movement controlling/compensating method and device of a surgical robot, which can change the relative position of a robot arm so as to be suitable for the surgical operation by the movement of the surgical robot without undocking the robot arm.

According to an aspect of the invention, there is provided a movement compensating device of a surgical robot in which a surgical operation processing unit mounted with a surgical instrument is coupled to one end of a body section, including: an image information creating unit that creates image information corresponding to an image signal supplied from a camera unit having captured an image of an operating site; a recognition point information analyzing unit that creates analysis information on a distance and an angle between a recognition point recognized from image information pieces corresponding to a predetermined number of image frames and a predetermined reference point; a variation analyzing unit that creates variation information in distance and angle between two analysis information pieces continuously created; and a control command creating and outputting unit that creates and outputs a control command for adjusting the position of the surgical operation processing unit so that the variation in distance and angle included in the variation information be 0 (zero).

The camera unit may be disposed at one end of the surgical operation processing unit.

A movement unit that allows the body section to move in any direction may be disposed under the body section.

The movement unit may include an omnidirectional wheel or may be embodied in the form of one or more of a magnetic levitation type and a ball wheel type.

Each recognition point may be an object which is included in the image frames so as to be recognized as an object by capturing an image of a recognition marker formed at one end of a medical trocar or a predetermined feature point to be included in the image information.

The surgical operation processing unit and one end of the body section may be coupled to each other through the use of a coupling unit, and the coupling unit may include a motor assembly that is adjusted to allow the surgical operation processing unit to rotate and to move in a horizontal direction in response to the control command.

According to another aspect of the invention, there is provided a surgical robot including: a movement unit that enables the surgical robot to move in any direction; a communication unit that receives a position shift command for causing the movement unit to move; and a movement processing unit that creates a control signal for causing the movement unit to move along a predetermined moving path in response to the position shift command.

The surgical robot may further include a storage unit that stores movement information on a moving direction and a moving distance of the movement unit so as to correspond to the position shift command, and the control signal may be a signal for causing the movement unit to move on the basis of the movement information corresponding to the position shift command.

The movement information may include information on the moving directions and the moving distances of movements between plural virtual path points included in the predetermined moving path.

The predetermined moving path may be drawn with a fluorescent dye on the floor or ceiling of an operating room so as to be recognized by a recognizer of the surgical robot and to move along the recognized moving path or may be formed in the form of a magnet or a magnetic rail under the floor of the operating room so as to induce the surgical robot to move.

The surgical robot may further include a sensor that senses the presence of an object coming near and that outputs a sensing signal, and the movement processing unit may output a stop command for stopping the movement of the movement unit to the movement unit or may stop creating and outputting the control signal for causing the movement unit to move, when the sensing signal is output from the sensor.

The movement unit may include an omnidirectional wheel or may be embodied in the form of one or more of a magnetic levitation type and a ball wheel type.

According to another aspect of the invention, there is provided a surgical robot including: a movement unit that enables the surgical robot to move in any direction; a communication unit that receives a position shift command for causing the movement unit to move; an external force detecting unit that determines whether an external force is applied to the surgical robot for the purpose of a moving operation using the movement unit; a movement processing unit that creates and outputs a movement control signal for causing the movement unit to move along a predetermined moving path to the movement unit in response to the position shift command when it is determined by the external force detecting unit that an external force is not applied; and a path resetting unit that resets the predetermined moving path for the movement corresponding to the position shift command when it is determined by the external force detecting unit that the external force is not applied any more.

When it is determined by the external force detecting unit that the external force is applied, the movement processing unit may stop creating and outputting the movement control signal until it is determined that the external force is not applied any more.

For the purpose of the resetting of the moving path, when the center point of an area of interest is not matched with the center point of a photographing area through the use of image information created to correspond to an image signal supplied from a camera unit having captured an image of an operating site, the path resetting unit may create and output a return control signal for causing the movement unit to move to the movement unit so as to enable the surgical robot to move to a position at which the center points are matched with each other.

When the area of interest is not recognized from the photographing area, the path resetting unit may create and output the return control signal for causing the movement unit to move so as to enable the surgical robot to move in the opposite direction of the direction in which the center point of the area of interest gets apart from the center point of the photographing area by an external force.

The path resetting unit may reset the moving path closest to the current position based on the external force out of plural predetermined moving paths as the moving path corresponding to the position shift command.

The surgical robot may further include a sensor that senses the presence of an object coming near and that outputs a sensing signal, and the movement processing unit may output a stop command for stopping the movement of the movement unit to the movement unit or may stop creating and outputting the control signal for causing the movement unit to move, when the sensing signal is output from the sensor.

The surgical robot may further include a storage unit that stores movement information on a moving direction and a moving distance of the movement unit so as to correspond to the position shift command, and the control signal may be a signal for causing the movement unit to move on the basis of the movement information corresponding to the position shift command.

The movement information may include information on the moving directions and the moving distances of movements between plural virtual path points included in the moving path.

The moving path may be drawn with a fluorescent dye on the floor or ceiling of an operating room so as to be recognized by a recognizer of the surgical robot and to move along the recognized moving path or may be formed in the form of a magnet or magnetic rail under the floor of the operating room so as to induce the surgical robot to move.

The movement unit may include an omnidirectional wheel or may be embodied in the form of one or more of a magnetic levitation type and a ball wheel type.

According to still another aspect of the invention, there is provided an operation unit performing a position shifting operation of a surgical robot, including: a display unit that displays image information captured with a ceiling camera unit; an input unit that is used to designate a destination position of the surgical robot with reference to the displayed image information; a storage unit that stores conversion reference information for the movement of the surgical robot from the current position to the destination position with reference to the image information; a movement information creating unit that creates position shifting information for causing the surgical robot to move to the destination position using the current position, the destination position, and the conversion reference information of the surgical robot; and a command creating unit that creates a position shift command corresponding to the position shifting information and supplies the created position shift command to the surgical robot.

The operation unit may further include a posture information creating unit that creates posture information for directing the front surface of the surgical robot to face an operating table or to face a side designated by a user and the command creating unit may further create a posture control command corresponding to the posture information and supply the created posture control command to the surgical robot.

The conversion reference information may be information used to convert the distance and angle between the current position and the destination position which are designated on the basis of the image information into a distance and an angle by which the surgical robot should move in the operating room.

The surgical robot may include: a movement unit that enables the surgical robot to move in any direction; a communication unit that receives a position shift command for causing the movement unit to move; and a movement processing unit that creates a control signal for causing the movement unit to move along a predetermined moving path in response to the position shift command.

The movement unit may include an omnidirectional wheel. The movement unit may be embodied in the form of one or more of a magnetic levitation type and a ball wheel type.

The operation unit may be mounted on a master robot coupled to the surgical robot via a communication network or may be an operation panel directly coupled to the surgical robot.

According to still another aspect of the invention, there is provided a surgical robot in which a surgical operation processing unit mounted with a surgical instrument is coupled to one end of a body section, including: a movement unit that enables the surgical robot to move in any direction; a storage unit that stores target rotating angle information corresponding to a position shift command for shifting the position of the surgical robot; a communication unit that receives rotating angle information based on analysis of an operating site image from a movement compensating device; and a movement processing unit that creates and outputs a control signal for causing the movement unit to move along a predetermined moving path to the movement so that a remainder rotating angle information obtained by subtracting the rotating angle information from the target rotating angle information be 0 (zero).

When movement information on a moving direction, a moving distance, and a rotating angle of virtual path points included in the moving path is stored in advance in the storage unit so as to correspond to the position shift command, the movement processing unit may determine whether the rotating angle information received from the movement compensating device is matched with the rotating angle included in the movement information within a margin of error and may stop the movement of the movement unit when they are not matched with each other within the margin of error.

The movement processing unit may update the remainder rotating angle information on the basis of the received total rotating angle information until the rotating angle of 0 (zero) is received from the movement compensating device and may then restart a process control of causing the movement unit to move along a moving path.

The movement compensating device may include: an image information creating unit that creates image information corresponding to an image signal supplied from a camera unit having captured an image of an operating site; a recognition point information analyzing unit that creates analysis information on a variation in angle between a recognition point recognized from image information pieces corresponding to a predetermined number of image frames and a predetermined reference point with respect to a predetermined reference line; a rotating angle calculating unit that calculates rotating angle information using the variation information in angle between two continuously-created analysis information pieces.

According to still another aspect of the invention, there is provided a surgical robot system having a surgical robot including a surgical operation processing unit mounted with a surgical instrument, including: a movement unit that is disposed in the surgical robot so as to enable the surgical robot to move in any direction; a tracking unit that recognizes the position of a recognition marker and that creates information on the moving direction and the moving distance of the surgical robot so as to cause the surgical robot to move to a designated target position; and a movement processing unit that creates and outputs a control signal for causing the movement unit to move on the basis of the moving direction and the moving distance determined from the created information.

The tracking unit may include one or more an optical tracker and a magnetic tracker.

The surgical robot system may further include a sensor that senses the presence of an object coming near and that outputs a sensing signal, and the movement processing unit may output a stop command for stopping the movement of the movement unit to the movement unit or may stop creating and outputting the control signal for causing the movement unit to move, when the sensing signal is output from the sensor.

The movement unit may include an omnidirectional wheel or may be embodied in the form of one or more of a magnetic levitation type and a ball wheel type.

According to still another aspect of the invention, there is provided a movement compensating device of a surgical robot in which a surgical operation processing unit mounted with a surgical instrument is coupled to one end of a body section, including: a tracking unit that creates analysis information on a distance and an angle between a recognition point which is a position of a recognition marker recognized by a predetermined number of image frames and a predetermined reference point and that creates variation information in the distance and the angle between two analysis information pieces continuously created; and a control command creating and outputting unit that creates and outputs a control command for adjusting the position of the surgical operation processing unit so that the variation in distance and angle included in the variation information be 0 (zero).

The tracking unit may be disposed at one end of the surgical operation processing unit and a movement unit that allows the body section to move in any direction may be disposed under the body section.

The recognition point may be a point indicating the position at which the recognition marker formed at one end of a medical trocar, the surgical operation processing unit and one end of the body section may be coupled to each other through the use of a coupling unit, and the coupling unit may include a motor assembly that is adjusted to allow the surgical operation processing unit to rotate and to move in a horizontal direction in response to the control command.

According to still another aspect of the invention, there is provided a movement compensating method of a surgical robot, which is performed by a movement compensating device, including: creating image information corresponding to an image signal supplied from a camera unit having captured an image of an operating site; creating analysis information on a distance and an angle between a recognition point recognized from image information pieces corresponding to a predetermined number of image frames and a predetermined reference point; creating variation information of the distance and the angle between two analysis information pieces continuously created; and creating and outputting a control command for adjusting the position of a surgical operation processing unit so that the variation in distance and angle included in the variation information be 0 (zero).

The surgical robot may include a body section and the surgical operation processing unit that is mounted with a surgical instrument and that is coupled to one end of the body section, and the camera unit may be disposed at one end of the surgical operation processing unit.

A movement unit that allows the body section to move in any direction may be disposed under the body section.

The movement unit may include an omnidirectional wheel or may be embodied in the form of one or more of a magnetic levitation type and a ball wheel type.

Each recognition point may be an object which is included in the image frames so as to be recognized as an object by capturing an image of a recognition marker formed at one end of a medical trocar or a predetermined feature point to be included in the image information.

The surgical operation processing unit and one end of the body section may be coupled to each other through the use of a coupling unit, and the coupling unit may include a motor assembly that is adjusted to allow the surgical operation processing unit to rotate and to move in a horizontal direction in response to the control command.

According to still another aspect of the invention, there is provided a position shifting method of a surgical robot having a movement unit that enables the surgical robot to move in any direction, including: receiving a position shift command for causing the movement unit to move; and creating a control signal for causing the movement unit to move along a predetermined moving path in response to the position shift command.

The position shifting method of a surgical robot may further include: determining whether a sensing signal is input from a sensor that senses the presence of an object coming near and that outputs the sensing signal; and outputting a stop command for stopping the movement of the movement unit to the movement unit or stopping creating and outputting the control signal for causing the movement unit to move, when the sensing signal is input.

When the predetermined moving path has a closed curve shape, the outputting of the stop command may include: calculating a moving distance in a clockwise direction and a moving distance in a counterclockwise direction as the moving distance from the current position to a position corresponding to the position shift command; and creating and outputting a control signal for causing the movement unit to move along the moving path in the moving direction corresponding to the relatively-short moving distance out of the calculated moving distances to the movement unit.

Movement information on the moving direction and the moving distance of the movement unit may be stored in a storage unit in advance so as to correspond to the position shift command, and the control signal may be a signal for causing the movement unit to move on the basis of the movement information corresponding to the position shift command.

The movement information may include information on the moving directions and the moving distances of movements between plural virtual path points included in the predetermined moving path.

The predetermined moving path may be drawn with a fluorescent dye on the floor or ceiling of an operating room so as to be recognized by a recognizer of the surgical robot and to move along the recognized moving path or may be formed in the form of a magnet or magnetic rail under the floor of the operating room so as to induce the surgical robot to move.

The movement unit may include an omnidirectional wheel or may be embodied in the form of one or more of a magnetic levitation type and a ball wheel type.

According to still another aspect of the invention, there is provided a moving path determining method of a surgical robot having a movement unit that enables the surgical robot to move in any direction, comprising: receiving a position shift command for causing the movement unit to move; determining whether an external force is applied to the surgical robot for the purpose of a moving operation using the movement unit; creating and outputting a movement control signal for causing the movement unit to move along a predetermined moving path to the movement unit to the movement unit in response to the position shift command when it is determined that the external force is not applied; and resetting the predetermined moving path for the movement corresponding to the position shift command when it is determined by the external force detecting unit that the external force is applied and then the application is stopped.

The resetting of the moving path may include: stopping creating and outputting the movement control signal when it is determined that an external force is applied; determining whether the application of an external force is maintained; resetting the predetermined moving path for the movement corresponding to the position shift command when the application of an external force is stopped; and creating and outputting a movement control signal for causing the movement unit to move along the reset moving path to the movement unit.

The resetting of the moving path may include: determining whether the center point of an area of interest is matched with the center point of a photographing area through the use of image information created to correspond to an image signal supplied from a camera unit having captured an image of an operating site when it is determined that an external force is not applied; and creating and outputting a return control signal for causing the movement unit to move to the movement unit so as to enable the surgical robot to move to a position at which the center points are matched with each other when it is determined that both center points are not matched with each other.

The outputting of the return control signal may include: determining whether the area of interest is recognized from the photographing area, creating and outputting the return control signal for causing the movement unit to move so as to enable the surgical robot to move in the opposite direction of the direction in which the center point of the area of interest gets apart from the center point of the photographing area by the external force when it is determined that the area of interest is not recognized; and creating and outputting the return control signal for causing the movement unit to move to the movement unit so as to enable the surgical robot to move a position at which both center points are matched with each other, when both center points are not matched and the area of interest is recognized from the photographing area.

According to still another aspect of the invention, there is provide a path returning method of a surgical robot, including: receiving a position shift command for causing a movement unit to move; and creating and outputting a movement control signal for causing the movement unit to move along a predetermined moving path in response to the position shift command to the movement unit when it is determined that an external force is not applied.

The path returning method of a surgical robot may further include: determining whether a sensing signal is input from a sensor that senses the presence of an object coming near and that outputs the sensing signal; and outputting a stop command for stopping the movement of the movement unit to the movement unit or may stop creating and outputting the control signal for causing the movement unit to move, when it is determined that the sensing signal is output from the sensor.

Movement information on a moving direction and a moving distance of the movement unit may be stored in advance in a storage unit so as to correspond to the position shift command, and the control signal may be a signal for causing the movement unit to move on the basis of the movement information corresponding to the position shift command.

The movement information may include information on the moving directions and the moving distances of movements between plural virtual path points included in the moving path.

The moving path may be drawn with a fluorescent dye on the floor or ceiling of an operating room so as to be recognized by a recognizer of the surgical robot and to move along the recognized moving path or may be formed in the form of a magnet or magnetic rail under the floor of the operating room so as to induce the surgical robot to move.

The movement unit may include an omnidirectional wheel or may be embodied in the form of one or more of a magnetic levitation type and a ball wheel type.

According to still another aspect of the invention, there is provided a position shifting method of a surgical robot which is performed by an operation unit, including: displaying image information captured with a ceiling camera unit; inputting a destination position of the surgical robot with reference to the displayed image information; and creating position shifting information for enabling the surgical robot to move to the destination position using conversion reference information stored in advance for the movement of the surgical robot from the current position to the destination position with reference to the image information and the current position and the destination position of the surgical robot and supplying the created position shifting information to the surgical robot.

The position shifting method may further include creating posture information for directing the front surface of the surgical robot to face an operating table or to face a side designated by a user and a posture control command corresponding to the created posture information may be further created and supplied to the surgical robot.

The conversion reference information may be information used to convert the distance and angle between the current position and the destination position which are designated on the basis of the image information into a distance and an angle by which the surgical robot should move in the operating room.

The surgical robot may include: a movement unit that enables the surgical robot to move in any direction; a communication unit that receives a position shift command for causing the movement unit to move; and a movement processing unit that creates a control signal for causing the movement unit to move along a predetermined moving path in response to the position shift command.

The operation unit may be mounted on a master robot coupled to the surgical robot via a communication network or may be an operation panel directly coupled to the surgical robot.

According to still another aspect of the invention, there is provided a position shifting method of a surgical robot having a movement unit that enables the surgical robot to move in any direction, including; storing target rotating angle information corresponding to a position shift command for shifting the position of the surgical robot; receiving rotating angle information based on analysis of an operating site image from a movement compensating device; and creating and outputting a control signal for causing the movement unit to move along a predetermined moving path to the movement so that a remainder rotating angle information obtained by subtracting the rotating angle information from the target rotating angle information be 0 (zero).

When movement information on a moving direction, a moving distance, and a rotating angle of virtual path points included in the moving path is stored in advance in the storage unit so as to correspond to the position shift command, the position shifting method may further include: determining whether the rotating angle information received from the movement compensating device is matched with the rotating angle included in the movement information within a margin of error; and stopping the movement of the movement unit when they are not matched with each other within the margin of error.

The position shifting method may further include: determining whether a rotating angle of 0 (zero) is received from a movement compensating device; updating the remainder rotating angle information on the basis of the total rotating angle information received after the movement of the movement unit is stopped when it is determined that a rotating angle of 0 is received, and restarting a process control of causing the movement unit to move along a moving path.

According to still another aspect of the invention, there is provided a position shifting method of a surgical robot which is performed in a surgical robot system, comprising: recognizing the position of a recognition marker; creating information on the moving direction and the moving distance of the surgical robot so as to enable the surgical robot to move to a designated target position; and creating and outputting a control signal for causing a movement unit of the surgical robot to move on the basis of the moving direction and the moving distance determined from the created information.

The tracking unit may include one or more an optical tracker and a magnetic tracker.

The position shifting method of a surgical robot may further include: determining whether a sensing signal is input from a sensor that senses the presence of an object coming near and that outputs the sensing signal; and outputting a stop command for stopping the movement of the movement unit to the movement unit or stopping creating and outputting the control signal for causing the movement unit to move, when the sensing signal is input from the sensor.

The movement unit may include an omnidirectional wheel or may be embodied in the form of one or more of a magnetic levitation type and a ball wheel type.

According to still another aspect of the invention, there is provided a movement compensating method of a surgical robot which is performed in a movement compensating device, including: creating analysis information on a distance and an angle between a recognition point which is a position of a recognition marker recognized by a predetermined number of image frames and a predetermined reference point; creating variation information in the distance and the angle between two analysis information pieces continuously created; and creating and outputting a control command for adjusting the position of the surgical operation processing unit so that the variation in distance and angle included in the variation information be 0 (zero).

The surgical robot may include a body section and a surgical operation processing unit coupled to one end of the body section and mounted with a surgical instrument and a tracking unit may be disposed at one end of the surgical operation processing unit.

A movement unit that enables the body section to move in any direction may be disposed under the body section and the recognition point may be a point indicating a position at which a recognition marker formed at one end of a medical trocar is recognized.

Other aspects, features, and advantages will be apparent from the accompanying drawings, the appended claims, and the below detailed description of the invention.

According to the above-mentioned aspects of the invention, it is possible to make a pre-process and a post-process of the movement of the surgical robot unnecessary by allowing the surgical robot to move to a desired position in a state where a surgical instrument and the like are inserted into a human body, thereby shortening the operating time and reducing a doctor's feeling of fatigue.

It is also possible to allow the surgical robot to freely move to a desired position only by inputting an operator's control command without an operator and/or an operation assistant's manual movement of the surgical robot to the desired position.

It is also possible to change the relative position of the robot arm so as to be suitable for the surgical operation by the movement of the surgical robot without undocking the robot arm.

### BRIEF DESCRIPTION OF THE DRAWINGS

FIG. 1 is a diagram schematically illustrating the configuration of a surgical robot according to an exemplary embodiment of the invention.

FIGS. 2A and 2B are diagrams illustrating examples of an omnidirectional wheel used for movement of a surgical robot according to an exemplary embodiment of the invention.

FIG. 3 is a diagram illustrating the appearance of a medical trocar according to an exemplary embodiment of the invention.

FIG. 4A is a block diagram illustrating the configuration of a movement compensating device according to an exemplary embodiment of the invention.

FIG. 4B is a diagram schematically illustrating a movement compensating method of the movement compensating device according to an exemplary embodiment of the invention.

FIGS. 5A to 5C are conceptual diagrams illustrating the behavior of the movement compensating device according to an exemplary embodiment of the invention.

FIG. 6 is a flowchart illustrating a movement compensating method according to an exemplary embodiment of the invention.

FIG. 7 is a diagram schematically illustrating the configuration of a body section of a surgical robot according to another exemplary embodiment of the invention.

FIG. 8A is a diagram illustrating a moving path of the surgical robot according to another exemplary embodiment of the invention.

FIG. 8B is a diagram illustrating control reference information of an omni-directional wheel according to another exemplary embodiment of the invention.

FIGS. 9A to 9C are conceptual diagrams illustrating the movement of the surgical robot according to another exemplary embodiment of the invention.

FIG. 10 is a flowchart illustrating a movement processing method of the surgical robot according to another exemplary embodiment of the invention.

FIG. 11 is a diagram schematically illustrating the configuration of a body section of a surgical robot according to still another exemplary embodiment of the invention.

FIG. 12 is a diagram illustrating a moving path of the surgical robot according to still another exemplary embodiment of the invention.

FIG. 13 is a diagram illustrating the concept of determining a return path of the surgical robot according to still another exemplary embodiment of the invention.

FIG. 14 is a flowchart illustrating a path returning method of the surgical robot according to still another exemplary embodiment of the invention.

FIG. 15 is a diagram schematically illustrating the configuration of a master robot according to still another exemplary embodiment of the invention.

FIG. 16 is a diagram illustrating an example of a screen display for causing the surgical robot according to still another exemplary embodiment of the invention to move.

FIG. 17 is a flowchart illustrating a movement processing method of the surgical robot according to still another exemplary embodiment of the invention.

FIG. 18 is a block diagram illustrating the configuration of a movement compensating device according to still another exemplary embodiment of the invention.

FIG. 19 is a conceptual diagram illustrating a movement compensating method of the movement compensating device according to still another exemplary embodiment of the invention.

FIG. 20 is a diagram illustrating an example of control reference information of an omni-directional wheel according to still another exemplary embodiment of the invention.

FIG. 21 is a diagram illustrating the concept of calculating a rotating angle according to still another exemplary embodiment of the invention.

FIG. 22 is a flowchart illustrating a movement processing method of the surgical robot according to still another exemplary embodiment of the invention.

FIGS. 23A to 23C are conceptual diagrams illustrating the movement of the surgical robot according to another exemplary embodiment of the invention.

### DETAILED DESCRIPTION

The invention can be modified in various forms and specific exemplary embodiments thereof will be described and shown in the drawings. However, the exemplary embodiments are not intended to limit the invention, but it should be understood that the invention includes all the modifications, equivalents, and substitutions belonging to the concept and technical scope of the invention. When it is determined that detailed description of known techniques associated with the invention can make the concept of the invention obscure, the detailed description will not be made.

Terms such as “first” and “second” can be used to describe various elements, but the elements are not limited to the terms. The terms are used only to distinguish one element from another element.

The terms used in the following description are used to merely describe specific exemplary embodiments, but are not intended to limit the invention. An expression of the singular number includes an expression of the plural number, so long as it is clearly read differently context. The terms such as “include” and “have” are intended to indicate that features, numbers, steps, operations, elements, components, or combinations thereof used in the following description are present, and it should be thus understood that the possibility of presence or addition of one or more different features, numbers, steps, operations, elements, components, or combinations thereof is not excluded.

Terms such as “section”, “-or/er”, “module”, and “unit” used in the following description means a unit performing at least one function or operation, and can be embodied by hardware, by software, or by a combination of hardware and software.

The exemplary embodiments of the invention will be described below in detail with reference to the accompanying drawings. In the below description with reference to the accompanying drawings, identical or corresponding elements are referenced by identical reference numerals and the description thereof will not be repeated.

Distinctive concepts of the exemplary embodiments will be mainly described with reference to the accompanying drawings. The invention is not limited to the independent exemplary embodiments, but one or more distinctive concepts of a certain exemplary embodiment may be added to another exemplary embodiment.

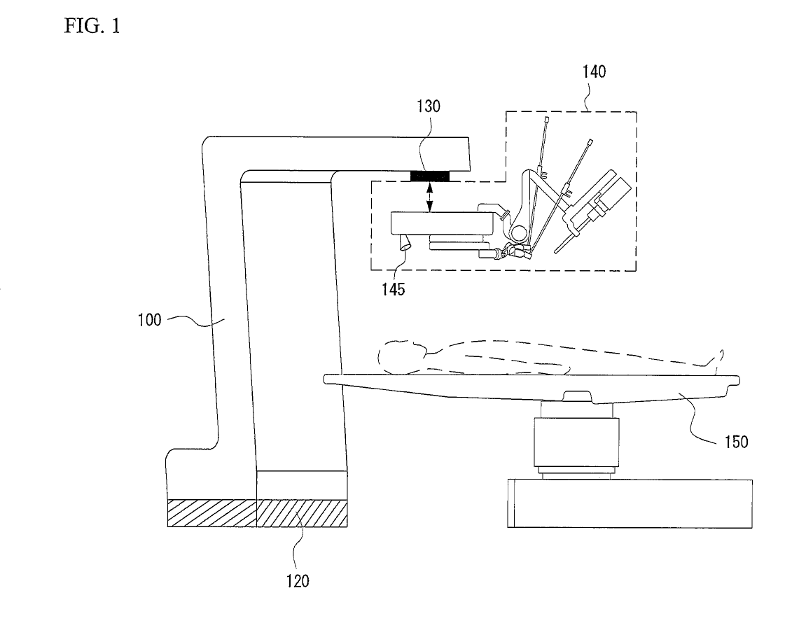

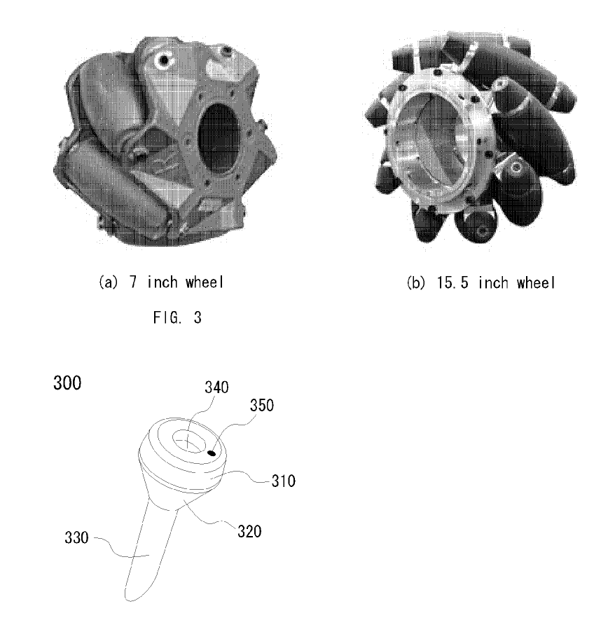

FIG. 1 is a diagram schematically illustrating the configuration of a surgical robot according to an exemplary embodiment of the invention. FIGS. 2A and 2B are diagrams illustrating examples of an omnidirectional wheel used for movement of a surgical robot according to an exemplary embodiment of the invention. FIG. 3 is a diagram illustrating the appearance of a medical trocar according to an exemplary embodiment of the invention.

The shapes of a surgical robot, an omnidirectional wheel, and a medical trocar shown in FIGS. 1 to 3 are examples used to describe an exemplary embodiment of the invention and the shapes or the like of the elements are not limited to the drawn shapes.

Referring to FIG. 1, a surgical robot includes a body section 100, a multi-directional wheel 120, a coupling unit 130, and a surgical operation processing unit 140.

The body section 100 is coupled to the surgical operation processing unit 140 and the like so as to perform a surgical operation on a patient on an operating table 150. The body section 100 may be a main body of a slave robot connected to a master robot via a communication network or a main body of a surgical robot into which a slave robot and a master robot are incorporated.

The multi-direction wheel 120 is coupled to the bottom of the body section 100 so as to move or rotate in any direction with an external force. The multi-direction wheel 120 enables the body section 100 to move with a force and a direction determined depending on the external force and may include an omni-directional wheel as shown in FIG. 2.

In this specification, it is assumed that a constituent element directly manipulated to enable the body section 100, that is, the surgical robot, to move is the multi-directional wheel 120, but the multi-directional wheel 120 may be embodied in the form of a magnetic levitation type or a ball wheel type. In this case, the multi-directional wheel 120 can be called a movement unit.

The surgical robot can be manipulated to actively move in response to a received control command even when an external force is not directly applied for the shift of a position.

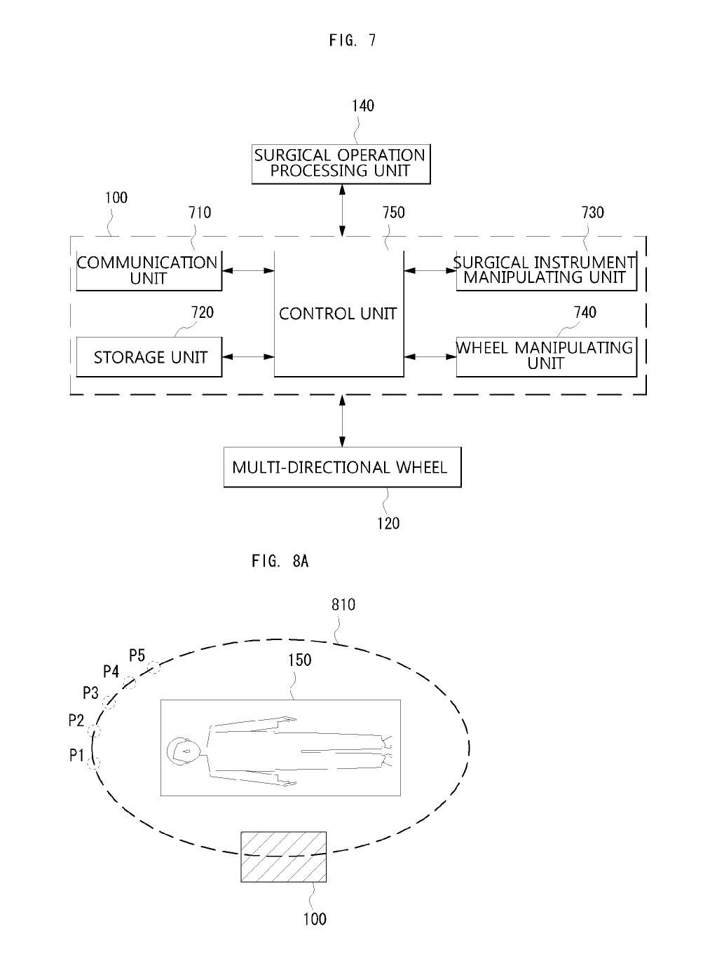

That is, the multi-directional wheel 120 can be manipulated to cause the surgical robot to move from a first position to a second position in a predetermined path in response to a position shift command (that is, a command to move from the first position as a current position to the second position as a destination position) received from a master robot (not shown, in which the master robot may be separated from the surgical robot or may be incorporated into the surgical robot). For this purpose, the body section 100 may further include a wheel manipulating unit 740 (see FIG. 7) that outputs a control command for causing the multi-directional wheel 120 to move along the predetermined path in response to the received position shifting direction.

The position shift command for causing the surgical robot to move may not be supplied from the master robot, but a control device for causing the surgical robot to move may be provided to the surgical robot or/and at a position close to the surgical robot in an operating room.

This is because it is more desirable to check the operating table 150 in the operating room and to cause the surgical robot to move than the case where the position shift command is received from the master robot apart from the operating table 150 to cause the surgical robot to move.

Various movement processing methods of causing the surgical robot to move can be employed, but it is assumed in this specification that the position shift command is transmitted from the master robot to the slave robot. However, this assumption does not limit the scope of the invention.

When the surgical robot departs from the predetermined path with a force directly from the outside while moving from the first position to the second position in response to the position shift command, the wheel manipulating unit 740 may output a control command for returning the surgical robot to the predetermined path to the multi-directional wheel 120 in response to a path returning command supplied from a return path determining unit 1130 (see FIG. 11).

The moving process of the surgical robot in response to the position shift command and/or the path returning command will be described in detail later with reference to the relevant drawings.

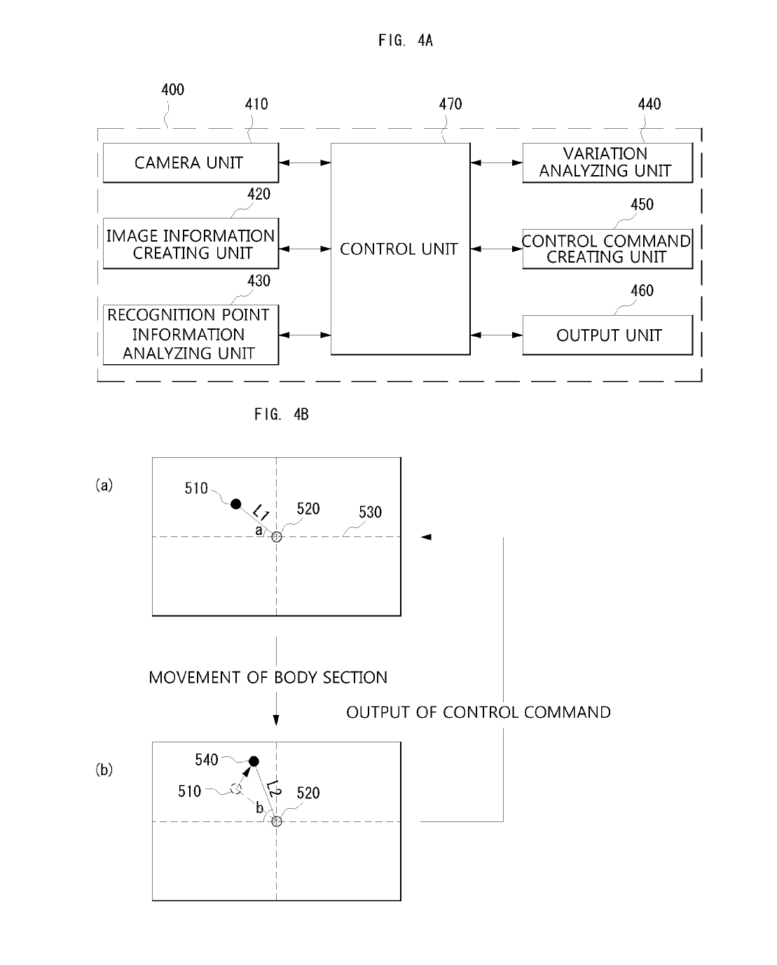

The coupling unit 130 couples the surgical operation processing unit 140 to one end of the body section 100 and the surgical operation processing unit 140 coupled thereto can move in all directions and/or rotate in the clockwise and counterclockwise directions in response to the control command input from a movement compensating device 400 (see FIG. 4A), when the body section 100 moves with the rotation and/or translation of the multi-directional wheel 120. Accordingly, an image input from a camera 145 can be kept constant regardless of the moving direction and angle of the body section 100 so as to enable the body section 100 to move in any direction. As a result, surgical instruments inserted into a human body can be continuously located at the same position within a margin of error regardless of the movement of the body section 100.

The coupling unit 130 may include an adjustment unit for the translational movement and the rotational movement so as to cause the surgical operation processing unit 140 to move in response to the control command. The adjustment unit may be embodied as a motor assembly for the translational movement and the rotational movement.

The configuration of the adjustment unit enabling to the surgical operation processing unit 140 to move in a rotational and/or translational moving manner in response to the input control command is obvious to those skilled in the art and thus description thereof will not be made.

The surgical operation processing unit 140 includes a robot arm and a surgical instrument (for example, one or more of instruments, laparoscope, and the like) coupled to or grasped by the robot arm and is coupled to an end of the body section 100 with the coupling unit 130. Although not shown in the drawings, the surgical operation processing unit 140 may include a vertical movement unit causing the surgical instrument to vertically move upward and/or downward.

The surgical operation processing unit 140 includes a camera 145 that creates image information of an operating site (for example, a position into which the surgical instrument is inserted through a medical trocar) based on the movement of the body section 100 and that supplies the created image information to the movement compensating device 400. As described later, the movement compensating device 400 checks the movement of the body section 100 using the image information supplied from the camera 145 and creates and outputs a control command for compensating for the movement (that is, causing the coupling unit 130 to move) so as to keep the image input from the camera 145 constant regardless of the movement of the body section 100.

FIG. 3 shows an appearance of a medical trocar 300 used to insert the surgical instrument into a human body.

As shown in the drawing, the medical trocar 300 includes an upper trocar housing 310, a lower trocar housing 320, a cannular 330, and a housing hole 340. Although not shown in the drawings, the medical trocar 330 may further include a discharge pipe used to discharge carcinogenic materials such as carbon monoxide and ammonia generated in the human body during the surgical operation. The cannular 330 is inserted into the human body through the skin of a site cut with a cutting tool such as a surgical knife and the surgical instrument (for example, one or more of instruments, laparoscope, and the like) is inserted into the human body via the housing hole 340 formed in the upper trocar housing 310 and the lower trocar housing 320 connected to the cannular 330.

A recognition marker 350 may be formed at one end of the upper trocar housing 310 of the medical trocar 300. The recognition marker 350 is photographed with the camera 145 and is recognized as a recognition point through the use of the image analysis of the movement compensating device 400. The recognition marker 350 may be formed, for example, in a figure of a predetermined color or with a fluorescent dye so as to facilitate the image analysis of the movement compensating device 400, or plural recognition markers may be formed at one or more positions of the upper trocar housing 310.

When a tracking device such as an optical tracker using infrared rays or a magnetic tracker using a magnetic technique is employed to track a position variation in addition to the camera 145, the recognition marker 350 may be a recognition marker for the tracking device.

The medical trocar 300 and the recognition marker 350 shown in FIG. 3 are on the assumption that the medical trocar is separated from the surgical robot and is fixed for the purpose of insertion of a surgical instrument into a human body. When the medical trocar 300 is coupled to the surgical robot, the medical trocar 300 moves along with the surgical robot and thus the recognition marker 350 cannot be used as the recognition point (see FIG. 4B) based on the movement of the surgical robot. In this case, a feature point (for example, a navel or an inner corner of an operating cover exposing only an operating site) fixed to an absolute position relative to a patient in operation in spite of the movement of the surgical robot can be used instead of the recognition marker 350.

FIG. 4A is a block diagram illustrating the configuration of the movement compensating device according to an exemplary embodiment of the invention and FIG. 4B is a diagram illustrating a movement compensating method of the movement compensating device according to the exemplary embodiment of the invention.

Referring to FIG. 4A, the movement compensating device 400 includes a camera unit 410, an image information creating unit 420, a recognition point information analyzing unit 430, a variation analyzing unit 440, a control command creating unit 450, an output unit 460, and a control unit 470. The movement compensating device 400 may be disposed in the body section 100 or the surgical operation processing unit 140 and gives a control command for causing the surgical operation processing unit 140 to move to the coupling unit 130. Although not shown in the drawings, the movement compensating device 400 may further include a storage unit that stores analysis information to be described later.

The camera unit 410 outputs an image signal created by capturing an image of an operating site (a position at which a surgical instrument is inserted into a human body via the medical trocar 300). The camera unit 410 may include, for example, an image sensor.

The camera unit 410 may be the same as the camera 145 described above with reference to FIG. 1. When the movement compensating device 400 is disposed in the body section 100, the camera unit 410 may be independent of the camera 145 of the surgical operation processing unit 140.

The image information creating unit 420 processes the image signal input from the camera unit 410 and creates image information to be output via a display device (not shown) disposed in or coupled to the master robot. The image information created by the image information creating unit 420 may have an image format of which the pixel information can be analyzed by the recognition point information analyzing unit 430. The image information creating unit 420 may include an image signal processor (ISP) performing one or more processes of a lens shading compensation process, a noise filter process, a flicker detection process, and an auto white balance process and a multimedia processor performing an image encoding/decoding process. The image format enabling an object included in the created image to be analyzed is obvious to those skilled in the art and thus description thereof will not be made.

The recognition point information analyzing unit 430 creates coordinate information of an object included in the image information created by the image information creating unit 420 and analysis information on the distance and angle relative to a reference point.

The object analyzed by the recognition point information analyzing unit 430 is the recognition marker 350 formed at one end of the upper trocar housing 310 of the medical trocar 300 described above with reference to FIG. 3 or a specific site (for example, a navel) of a patient, or a specific site of an operating cover, or the like. That is, the recognition point information analyzing unit 430 extracts an outline of the recognition marker from the image created by the image information creating unit 420 through the use of an image processing technique, recognizes the center point (that is, a recognition point 510 (see FIG. 4B)) of the extracted outline, and analyzes the coordinate information of the recognition point 510. Here, the analyzed coordinate information may be, for example, a relative coordinate with respect to the leftmost and lowermost point of the image as (0, 0).

The reference point may be a predetermined point in the image created by the image information creating unit 420. It is assumed that in this specification that the center point (that is, a screen center point 520 (see FIG. 4B) which is the center point of the display screen) in the horizontal and vertical directions of the display screen on which the image is displayed is the reference point, but the reference point is not limited to this case. The coordinate of the screen center point 520 may be designated in advance and may not be changed.

The recognition point information analyzing unit 430 creates analysis information including calculated distance L1 an angle a between the recognition point 510 and the screen center point 520. The reference line used to calculate the angle between the recognition point 510 and the screen center point 520 can be variously set and the horizontal line is defined as the reference line in this specification.

The recognition point information analyzing unit 430 creates analysis information pieces of a predetermined number of image frames out of image frames sequentially created by the image information creating unit 420. For example, the recognition point information analyzing unit 430 may create analysis information pieces of all the image frames sequentially created on the basis of a predetermined criterion, or may create analysis information pieces of the even image frames (the second image frame, the fourth image frame, and the like).

The variation analyzing unit 440 creates variation information on the distance and the angle between the analysis information pieces created for the image frames by the recognition point information analyzing unit 430.

FIG. 4B shows a position shift of the recognition points 510 and 540 in a first image frame and a second image frame due to the movement of the body section 100.

The number of recognition points used to create the variation information may be one or more. Two or more recognition points may be used to recognize the distance variation and the rotating angle due to the movement of the recognition point.

However, even when only one recognition point is designated, the distance variation and the rotating angle can be recognized by analyzing the relationship between the screen center point 520 which is a recognition reference point and one recognition point 510 or 540, as described later. In this case, by using the screen center point 520 as an invariable recognition reference point which will not be changed, the variation information of the distance variation and the rotating angle due to the movement of the recognition points 510 and 540 may be more accurate. Here, it is assumed that the screen center point 520 is effectively used as a fixed reference point regardless of the positional change of the recognition points 510 and 540 in the image created by the image information creating unit 420. However, when the screen center point 520 is not effective as the reference point due to the positional change of the recognition points 510 and 540 or the like, a reference point correcting process (for example, a correcting process of matching the screen center point with a specified reference point) of effectuating the screen center point 520 as a reference point may further performed. The reference point setting and correcting process of calculating the moving (rotating) direction and the moving distance of the recognition point changed in position is obvious to those skilled in the art and thus description thereof will not be made.

First, the recognition point information analyzing unit 430 creates the analysis information of the distance L1 and the angle a between the first recognition point 510 and the screen center point 520 in the first image frame shown in (a) of FIG. 4B.

Then, the recognition point information analyzing unit 430 creates the analysis information of the distance L2 and the angle b between the second recognition point 540 and the screen center point 520 in the second image frame shown in (b) of FIG. 4B. In this case, the screen center point 520 means a middle point of the entire screen area even when a subject image input from the camera is changed, and thus is present at a fixed position regardless of the positional change of the recognition points 510 and 540. The variation analyzing unit 440 creates the variation information using the analysis information pieces created for the first image frame and the second image frame. The variation information may include the variation in distance L2-L1 and the variation in angle b-a and it is analyzed that the body section 100 moves by the absolute value of the variation.

The surgical operation processing unit 140 also moves to correspond to the movement of the body section 100 and the camera 145 included in the surgical operation processing unit 140 also moves to correspond thereto. In this case, the image captured with the movement of the camera 145 is displayed as if it moves in the opposite direction of the moving direction of the body section 100. Accordingly, it is analyzed that the body section 100 moves by (−1) times the variation.

The control command creating unit 450 creates a control command for controlling the coupling unit 130 so that the surgical operation processing unit 140 is located at the position at which the variation information created by the variation analyzing unit 440 becomes 0, that is, at a position at which the second recognition point 540 is matched with the first recognition point 510.

The control command serves to cause the body section to move in the translational and/or rotational moving manner in the direction and by the distance by which the position of the recognition point is fixedly maintained with the movement of the coupling unit 130 (that is, by which the variation information of the surgical operation processing unit 140 is 0). The position of the surgical operation processing unit 140 can be kept at the position before the body section 100 moves by the adjustment of the coupling unit 130 corresponding to the control command, even when the body section 100 moves in any direction.

The output unit 460 outputs the control command created by the control command creating unit 450 to the coupling unit 130 so as to keep the image input from the camera unit 410 constant. The constant image input from the camera unit 410 means that the position of the surgical operation processing unit 140 relative to the patient on the operating table 150 is kept constant.

The output unit 460 also transmits the control command to the master robot so as to recognize the operation of the coupling unit 130 of keeping the position of the surgical operation processing unit 140 constant. The output unit 460 may transmit the control command to the master robot so as to output the image information created by the image information creating unit 420 on the display device (not shown) disposed in or coupled to the master robot.

The control unit 470 controls the constituent element of the movement compensating device 400 to perform the above-mentioned functions.

Hitherto, the method of processing the movement of the coupling unit 130 using the variation of the analysis information on the distance and the angle between one recognition point and one reference point (for example, the screen center point) has been described.

However, when plural medical trocars 300 are inserted into a human body through the skin of a patient, the medical trocars 300 are separated from the surgical robot, and the recognition marker 350 is formed in each medical trocar 300, the center points of virtual lines connecting the recognition points as the recognition markers 350 may be located at the screen center and the position of the surgical operation processing unit 140 may be adjusted using the analysis information on the distances and the angles between the reference point as the center point located at the screen center and the recognition points and the variation information based thereon.

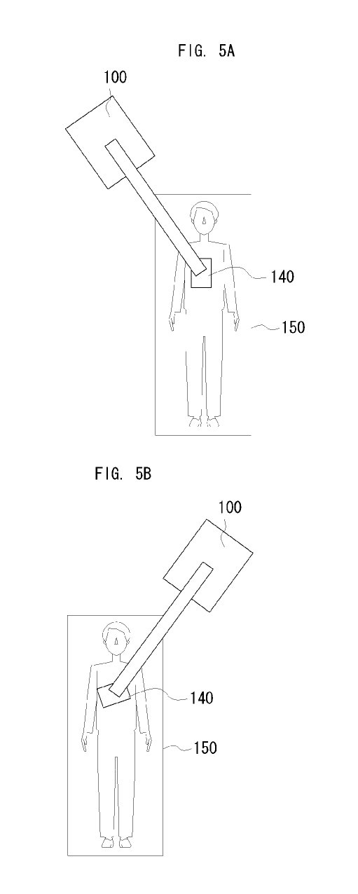

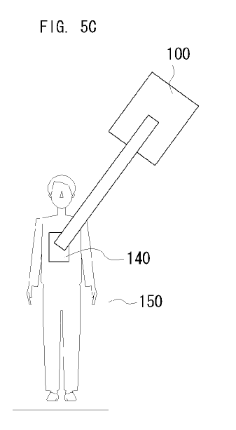

FIGS. 5A to 5C are conceptual diagrams illustrating the operation of the movement compensating device according to an exemplary embodiment of the invention.

That is, FIGS. 5A to 5C are diagrams illustrating the relationship between a patient and the body section 100, the surgical operation processing unit 140, and the operating table 150 before and after the body section 100 moves. For the purpose of simplification of the drawings, the instrument and the like included in the surgical operation processing unit 140 are not shown in the drawings.

It is assumed that the body section 100 should be made to move from the first position (that is, the right side of the patient's head) shown in FIG. 5A to the second position (that is, the left position of the patient's head) shown in FIGS. 5b and 5C. In a surgical robot according to the related art, the surgical operation processing unit 140 faces a position other than the original position as shown in FIG. 5B. To prevent an accident which may occur in this case, the surgical robot according to the related art requires a work of undocking all the robot arms, moving, and re-docking the robot arms.

However, in the surgical robot according to the exemplary embodiment of the invention, the position and direction of the surgical operation processing unit 140 is fixed relative to the patient by the function of the movement compensating device 400 as shown in FIG. 5C, even when the body section 100 moves from the first position to the second position.

In this case, the movement and/or rotation of the coupling unit 130 under the control of the movement compensating device 400 are performed through the use of a method of recognizing the reference point (for example, the screen center point) in the image process and checking how the recognition points 510 and 540 are changed relative to the reference point to calculate the variation and the like, as described above with reference to FIG. 4B.

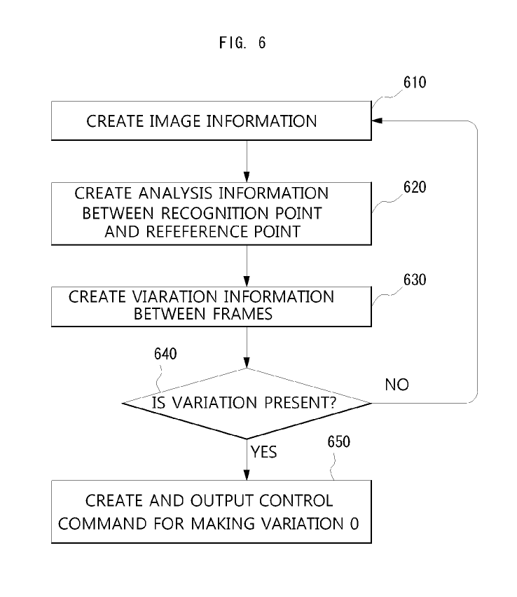

FIG. 6 is a flowchart illustrating a movement compensating method according to an exemplary embodiment of the invention.

Referring to FIG. 6, the movement compensating device 400 creates image information corresponding to an image signal supplied from the camera unit 410 in step 610.

In step 620, the movement compensating device 400 creates analysis information corresponding to the distance and the angle between the recognition point and the reference point using the image information. Here, the analysis information may be created only for image frames specified to create variation information to be described later.

In step 630, the movement compensating device 400 creates the variation information of the distance and the angle between the analysis information pieces of the image frames specified to create the variation information.

In step 640, the movement compensating device 400 determines whether a variation is present in the variation information (that is, whether the variation is 0 (zero)).

When it is determined that the variation is not present, the process of step 610 is performed again.

However, when it is determined that the variation is present, the movement compensating device 400 creates a control command for making the variation 0 and outputs the created control command to the coupling unit 130 in step 650. With the output of the control command for making the variation 0, the coupling unit 130 controls the surgical operation processing unit 140 so that the position of the surgical operation processing unit 140 is kept constant relative to the patient on the operating table 150 (that is, so that the image input from the camera unit 410 is kept constant).

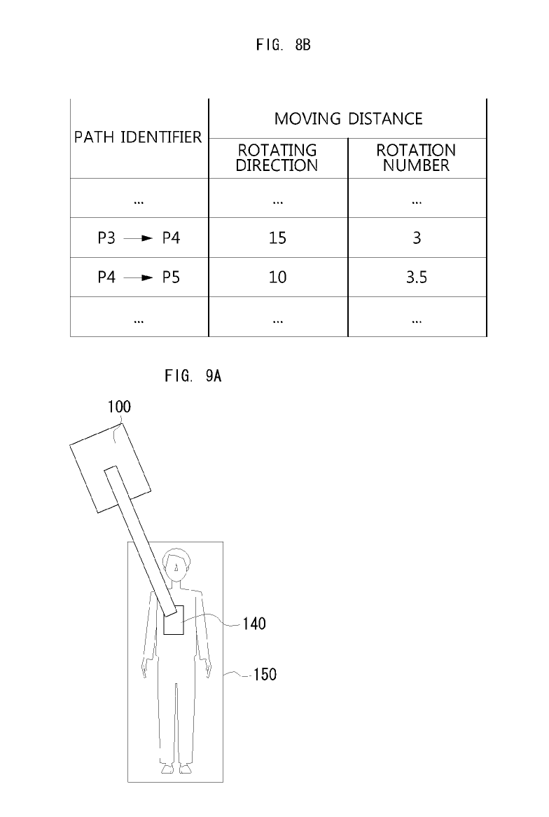

FIG. 7 is a diagram schematically illustrating the configuration of a body section of a surgical robot according to another exemplary embodiment of the invention. FIG. 8A is a diagram illustrating a moving path of the surgical robot according to another exemplary embodiment of the invention. FIG. 8B is a diagram illustrating control reference information of the multi-directional wheel according to another exemplary embodiment of the invention.

Referring to FIG. 7, the body section 100 includes a communication unit 710, a storage unit 720, a surgical instrument manipulating unit 730, a wheel manipulating unit 740, and a control unit 750.

Although not shown in the drawings, the body section 100 may further include a proximity sensor that senses a distance from the operating table 150 or the like so as not to collide with the operating table 150 or other obstacles during the movement along a moving path 810 to be described later. Here, the proximity sensor may be embodied in a detection type based on a mechanical contact (such as a micro switch and a limit switch) or a non-contact detection type (such as a high-frequency oscillating proximity sensor using energy loss of an induced current and an electrostatic proximity sensor using a variation in electrostatic capacitance due to a polarization phenomenon).

As described above, the surgical operation processing unit 140 can be made to move in all directions and/or to rotate in the clockwise direction and counterclockwise direction in response to the control command input from the movement compensating device 400 during the movement of the surgical robot to be described with reference to FIG. 7 or the like.

The communication unit 710 receives a control command (such as a position shift command and a surgical instrument manipulating command) from the master robot or transmits the image information supplied from the camera unit 410 to the master robot.

The storage unit 720 stores one or more of an operating program for performing the functions of the body section 100 and control commands received from the master robot. The storage unit 720 may further store control reference information for manipulating the multi-directional wheel 120 in response to the position shift command received from the master robot.

The control reference information stored in the storage unit 720 may be information on the rotating direction (that is, the moving direction of the body section 100) and the rotation number (that is, the moving distance of the body section 100) of the multi-directional wheel 120 for movement between the virtual path points as shown in FIG. 8B. The information is used by the wheel manipulating unit 740 controlling the multi-direction wheel 120 so as to cause the multi-directional wheel to move the destination position information (which can be designated by an operator) included in the position shift command. The control reference information stored in advance for the movement of the body section 100 is not limited to the information shown in FIG. 8B, but may be set to various formats so as to enable the body section 100 to move along a predetermined moving path 810.

The surgical instrument manipulating unit 730 creates a control signal for manipulating the surgical instrument of the surgical operation processing unit 140 (for example, changing the position of an endoscope or cutting the operating site) in response to the surgical instrument manipulating command received from the master robot and outputs the created control signal to the surgical operation processing unit 140.

The wheel manipulating unit 740 creates a control signal for causing the multi-direction wheel 120 to rotate in the corresponding direction and moving distance in response to the position shift command received from the master robot and outputs the created control signal to the multi-directional wheel 120.

When a sensing signal indicating that the operating table 150 or a peripheral obstacle comes near is received from the proximity sensor during the movement along the moving path 810, the wheel manipulating unit 740 may output a stop command for stopping the movement of the multi-directional wheel 120 to the multi-directional wheel 120 or may stop creating and outputting the control signal for manipulating the multi-directional wheel 120.

The control unit 750 controls the constituent elements of the body section 100.

FIG. 8A shows the moving path 810 of the surgical robot relative to the operating table 150.

The moving path 810 of the surgical robot may be embodied by the sequence of one or more virtual path points Px (P1, P2, and the like) and the virtual path points may be arranged continuously or discretely.

The surgical robot moves from the current position to the destination position via the virtual path points arranged in the moving path in response to the position shift command (which includes the destination position information or the information on the virtual path point corresponding to the destination position) received from the master robot.

The moving path 810 may be drawn on the floor or ceiling of the operating room centered on the operating table 150 with a fluorescent dye which can be recognized by the surgical robot.

In this case, the surgical robot may further include a camera (not shown) disposed at a position (for example, a lower area of the multi-directional wheel 120 or an upper area of the body section 100) corresponding to the position (such as the floor or the ceiling) in the operating room in which the moving path is drawn. The camera captures an image of the shown moving path 810 and supplies the captured image to the body section 100. The body section 100 analyzes the moving path 810 from the image information supplied from the camera through the use of an image analysis technique and creates and outputs a control signal for controlling the multi-directional wheel 120 to move along the moving path 810.

In another example, the moving path 810 may be formed as a magnet and/or a magnetic rail embedded under the floor of the operating room relative to the operating table 150. The body section 100 may create and output a control signal for causing the multi-directional wheel 120 to be induced by the magnet and the like embedded under the floor of the operating room and to move along the moving path 810. For example, a method of causing an electric cart to move along a designated cart road by the use of a remote controller in a golf course can be similarly used as the method of embedding a magnet or the like under the floor of the operating room to induce the multi-directional wheel 120.

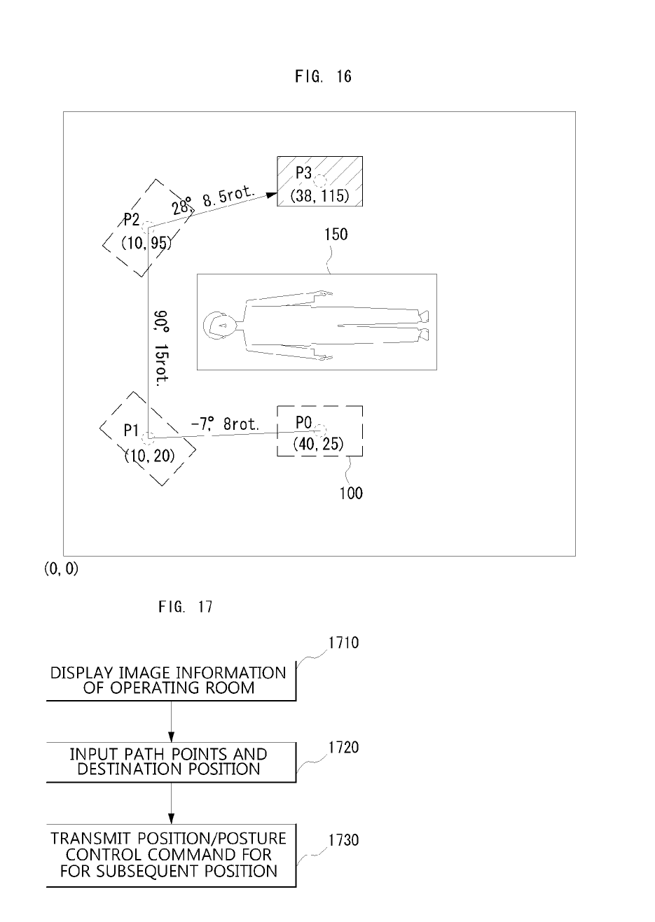

Even when the moving path 810 is not embodied by the fluorescent dye or the magnetic rail, the surgical robot may move in consideration of the relative position. Some examples of the movement in consideration of the relative position will be specifically described with reference FIGS. 8B and 15.

For example, an optical tracker, a magnetic tracker, or other tracking techniques may be used to determine the relative position of the surgical robot and the operating table 150. That is, when the optical tracking or the like is disposed at a specific position in the operating room and a recognition marker (such as an optical marker) is formed in the surgical robot and the operating table 150 (and/or a patient), a path in which the surgical robot does not collide with the operating table 150 or other objects may be generated and the surgical robot may move to the designated destination, without causing the surgical robot to move along the predetermined moving path 810 as described above.

By installing a camera in the surgical robot or on the ceiling of the operating room and processing and analyzing the image of the operating table 150 and/or the patient supplied from the camera, a method of causing the surgical robot to a destination via a path other than the predetermined moving path 810 may be employed. The example where the surgical robot moves to a destination using the image supplied from the camera installed on the ceiling of the operating room will be described in detail with reference to the relevant drawings.

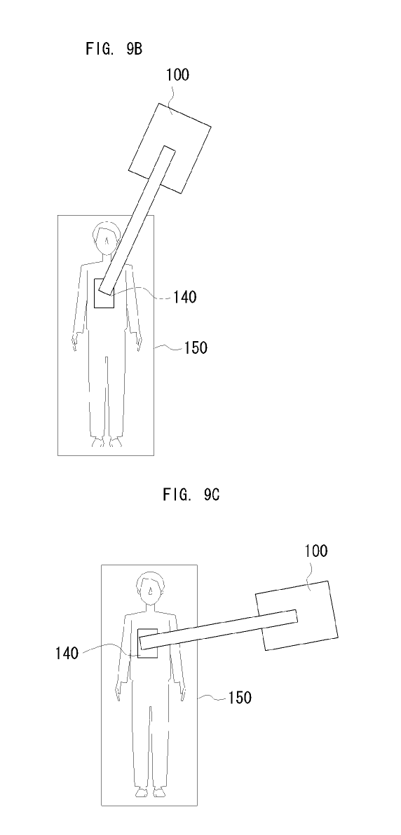

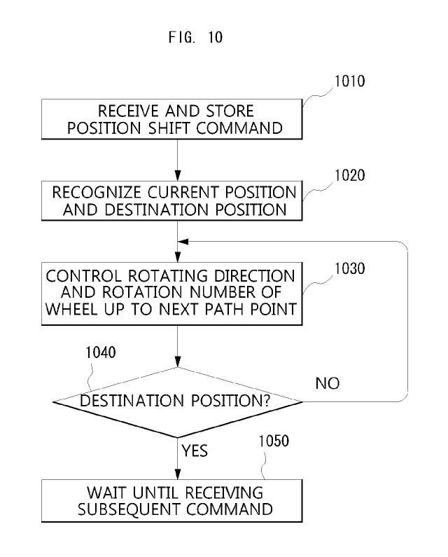

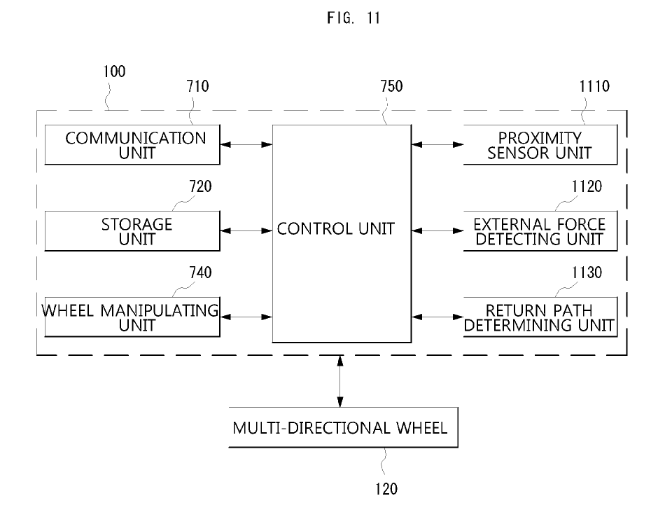

In the examples of the movement of the surgical robot, the multi-directional wheel 120 is controlled appropriately on the basis of the moving direction and the moving distance and the coupling unit 130 is controlled appropriately as needed (see FIGS. 9A to 9C).

FIG. 8B shows the control reference information for causing the body section 100 to move along the predetermined moving path 810.

As described above, the moving path 810 of the surgical robot is formed by the sequence of one or more virtual path points Px (for example, P1, P2, . . . ) and the virtual path points may be arranged continuously or discretely.

The control reference information stored in advance in the storage unit 720 may include the information on the rotating direction (that is, the moving direction of the body section 100) and the rotation number (that is, the moving distance of the body section 100) of the multi-directional wheel 120 for the movement between the virtual path points. For example, information on the moving distance between the virtual path points such as information that the multi-directional wheel 120 should be made to rotate by three rotations in a direction inclined about a predetermined reference line (for example, the horizontal straight line in the operating room) for the movement from the virtual path point P3 to the virtual path point P4 may be stored in the storage unit 720 in advance.

In this way, when the body section 100 controls the multi-directional wheel 120 on the basis of the control reference information stored in advance, the body section 100 can move along the predetermined moving path 810. However, since the body section 100 manipulates the multi-directional wheel 120 to the destination position sequentially via the virtual path points located in the moving path on the basis of the control reference information stored in advance, the body section 100 needs to be located in the predetermined moving path at the time of starting the movement. For this purpose, the moving path may be designated and drawn on the floor of the operating room.

FIGS. 9A to 9C are diagrams illustrating the concept of the surgical robot according to another exemplary embodiment of the invention.

That is, FIGS. 9A to 9C are diagrams illustrating the relationship between a patient and the body section 100, the surgical operation processing unit 140, and the operating table 150 before and after the body section 100 moves. For the purpose of simplification of drawing, the instrument and the like included in the surgical operation processing unit 140 are not shown.

As shown in FIGS. 9A to 9C, when the body section 100 is intended to move from the right side of the patient's head to the left position of the patient's head, the body section 100 sequentially moves to the position shown in FIGS. 9B and 9C by controlling the behavior of the multi-directional wheel 120.

In this case, as can be seen in the drawings, the surgical operation processing unit 140 is present at a fixed position and in a fixed direction relative to the patient. Accordingly, the coupling unit 130 coupled to the surgical operation processing unit 140 can be appropriately controlled with the control of the multi-directional wheel 120 during the movement of the body section 100. That is, the multi-directional wheel 120 and the coupling unit 130 can be automatically appropriately controlled so as not to change the relative position between the surgical operation processing unit 140 and the patient.

By using this composite control method, it is possible to change the relative position of the robot arm to be suitable for the operation without undocking the robot arm. It is also possible to avoid troubles such as the undocking and re-docking of the robot arm which are problems in the movement of the surgical robot according to the related art.

As described as various examples in this specification, the method of causing the body section 100 to move along the predetermined moving path 810, the method of designating the final destination position through the use of a graphic interface using an image input from a camera and causing the body section 100 to move thereto, the method of causing the body section 100 to move in response to the control command transmitted from the master robot or the movement command input through a control device installed in the body section 100, and the like can be used to cause the body section 100 from the first position to the second position. It will be obvious to those skilled in the art that other methods not described in this specification can be used to cause the body section 100 to move without any restriction.

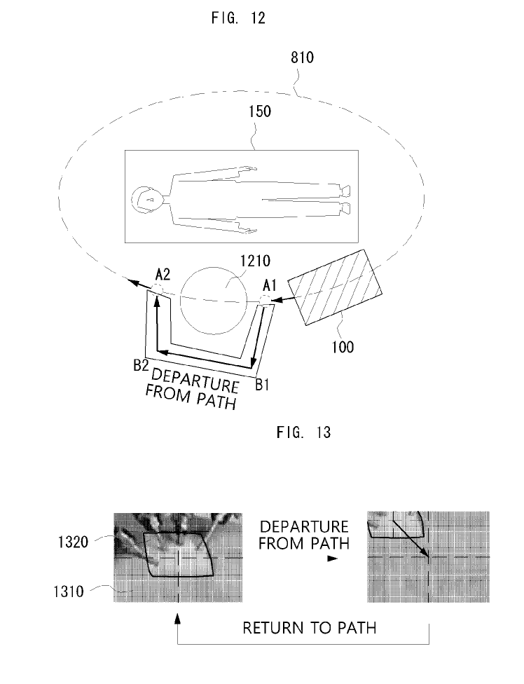

FIG. 10 is a flowchart illustrating a movement processing method of a surgical robot according to another exemplary embodiment of the invention.

Referring to FIG. 10, the body section 100 receives a position shift command from the master robot and stores the received position shift command in the storage unit 720 in step 1010. The position shift command includes at least destination position information.

In step 1020, the body section 100 recognizes the current position of the surgical robot and the destination position included in the position shift command. The body section 100 can recognize the current position and the destination position, for example, using the information of the virtual path points arranged in the moving path.

The body section 100 may set the moving direction (for example, the clockwise direction or the counterclockwise direction) of the movement along the moving path in advance using the recognized current position and the destination position or may determine the moving direction in real time.

For example, the moving distances in various directions in which the body section moves from the first virtual path point to the eighth virtual path point as the destination position may be determined and the direction in which the moving distance is smaller may be determined as the moving direction. At this time, since the moving path 810 is set in advance, the direction in which the moving distance is the smallest can be easily determined on the basis of the current position and the destination position.

In step 1030, the body section 100 creates and outputs the control signal for controlling the multi-directional wheel 120 to move to a subsequent virtual path point located in the moving path.

As described above, the body section 100 may create the control signal by referring to the image information of the moving path drawn with a fluorescent dye, using the magnet and/or magnetic rail embedded in the floor of the operating room so as to induce the movement of the surgical robot, or by using the control reference information stored in the storage unit 720 in advance.

In step 1040, the body section 100 determines whether the current position reached under the control of the multi-directional wheel 120 in step 1030 is the destination position corresponding to the position shift command. For example, the determination may be carried out depending on whether the virtual path point corresponding to the current position is matched with the virtual path point corresponding to the destination position.

When it is determined in step 1040 that the current position is not the destination position, the process of step 1030 is performed again.

However, when it is determined in step 1040 that the current position is the destination position, the body section 100 waits at the current position until a new command (for example, one or more of the surgical instrument manipulating command and the position shift command) is received from the master robot.

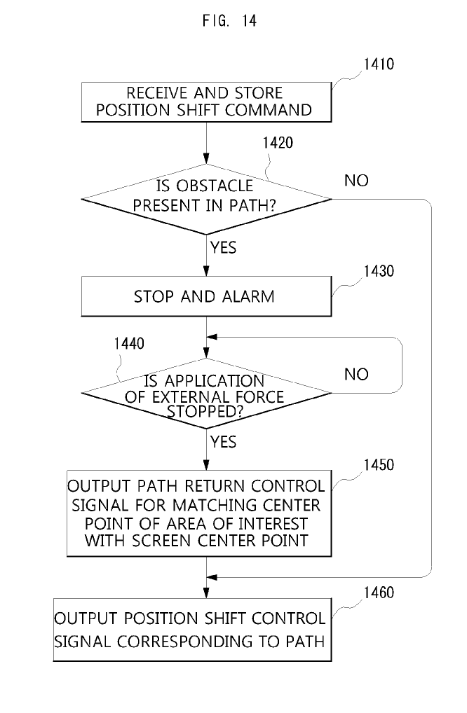

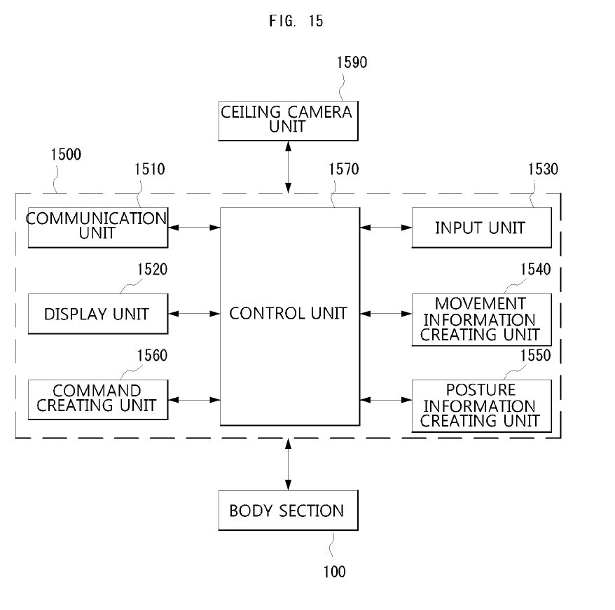

FIG. 11 is a diagram schematically illustrating the configuration of the body section of a surgical robot according to still another exemplary embodiment of the invention, FIG. 12 is a diagram illustrating the moving path of the surgical robot according to still another exemplary embodiment of the invention, and FIG. 13 is a diagram illustrating the concept of return path determination of the surgical robot according to still another exemplary embodiment of the invention.

Referring to FIG. 11, the body section 100 includes a communication unit 710, a storage unit 720, a proximity sensor unit 1110, an external force detecting unit 1120, a return path determining unit 1130, a wheel manipulating unit 740, and a control unit 750. Although not shown in the drawing, the body section 100 may further include an alarm unit that gives an alarm in a visual type and/or an auditory type when sensing an obstacle during the movement along the moving path 810.

The communication unit 710 receives a control command from the master robot or transmits image information supplied from the camera unit 410 to the master robot.

The storage unit 720 stores one or more of an operating program for performing the functions of the body section 100, control commands received from the master robot, and the control reference information for driving the multi-directional wheel 120.

The proximity sensor unit 1110 creates and outputs a sensing signal indicating the distance from an object located in the proximity. The proximity sensor unit 1110 includes a proximity sensor. The proximity sensor creates a distance sensing signal so that the body section 100 should not collide with the operating table 150 and/or the obstacle located in the moving path 810 during the movement along the moving path 810. The proximity sensor may be embodied in a detection type based on a mechanical contact (such as a micro switch and a limit switch) or a non-contact detection type (such as a high-frequency oscillating proximity sensor using energy loss of an induced current and an electrostatic proximity sensor using a variation in electrostatic capacitance due to a polarization phenomenon).

The external force detecting unit 1120 determines whether an external force is applied to cause the surgical robot to move. Here, an external force may include a force directly applied to the surgical robot so as to change the moving path or the like by an operator or an operator assistant, a force applied to the surgical robot so as to cause the surgical robot to move and/or a force applied to a position close to the surgical robot in the operating room so as to change the moving path through the use of the control device for the movement of the surgical robot, and a force applied to depart from the current moving path in response to a movement command, which is received from the master robot or input through the control device by the operator or the like during the movement of the surgical robot described above with reference to FIGS. 9A to 9C, so as to change the moving path. For the purpose of easy explanation and understanding, it is assumed that the force applied directly to the surgical robot by the operator or the operator assistant is defined as the external force.

For example, when the proximity sensor unit 1110 senses an obstacle in the moving path during the movement of the surgical robot along the moving path 810, the wheel manipulating unit 740 controls the multi-directional wheel 120 so as to stop (that is, pause) the movement of the surgical robot. At this time, the alarm unit (not shown) gives an alarm in the visual type (for example, the flickering of an LED) and/or an auditory type (the output of a warning sound).

In this way, depending on whether the multi-directional wheel 120 is manipulated to rotate with an application of an external force in the state where the movement of the surgical robot is stopped, the external force detecting unit 1120 can determined whether an external force is applied. A sensor sensing the rotation of the multi-directional wheel 120 may be further provided. The external force detecting unit 1120 can monitor the presence of an external force, even when it is determined that an obstacle is not present by the use of the sensing signal of the proximity sensor unit 1110 and the multi-directional wheel 120 is being controlled by the use of the wheel manipulating unit 740.

When an external force is applied during the movement of the surgical robot along the predetermined moving path 810 in response to the position shift command received from the master robot, thus the movement is stopped, and it is determined by the external force detecting unit 1120 that the application of the external force is stopped, the return path determining unit 1130 determines the moving direction and the moving distance of the surgical robot so that the surgical robot is returned to the moving path 810 using the image information supplied from the movement compensating device 400. FIG. 12 shows only one predetermined moving path 810, but plural moving paths may be set in advance. The wheel manipulating unit 740 should control the multi-directional wheel 120 so as to respond to the path return command corresponding to the moving direction and the moving distance determined by the return path determining unit 1130.

The return path determining unit 1130 may determine the moving direction and the moving distance using an optical tracker, a magnetic tracker, or other position trackers, as well as using the image information supplied from the movement compensating device 400. For example, by installing a tracker at a specific position of the operating room and positioning a recognition marker in the body section 100 or/and the surgical operation processing unit 140, the position of the surgical robot can be recognized and the moving direction or the like can be determined.

The wheel manipulating unit 740 creates a control signal for rotationally driving the multi-directional wheel 120 in the corresponding direction by the moving distance in response to the position shift command received from the master robot and outputs the created control signal to the multi-directional wheel 120.

The wheel manipulating unit 740 stops the movement of the surgical robot when an obstacle is sensed by the proximity sensor unit 1110 during the movement of the surgical robot along the moving path 810 or an external force is detected by the external force detecting unit 1120, and controls the operation of the multi-directional wheel 120 on the basis of the moving direction and the moving distance determined by the return path determining unit 1130 when the external force is not detected by the external force detecting unit 1120.

The control unit 750 controls the functions of the constituent elements of the body section 100.

FIG. 12 shows the moving path of the surgical robot and FIG. 13 shows the concept of the return path determination of the surgical robot.

As shown in FIG. 12, when an obstacle is sensed during the movement of the body section 100 (that is, the surgical robot) along the moving path in the direction of the shown arrow, the body section 100 stops the movement at a virtual path point A1. At this time, the alarm unit may give an alarm in the visual type or/and an auditory type.

Thereafter, a user such as an operator applies an external force to the surgical robot so as to avoid the obstacle and shifts the surgical robot to the positions B1 and B2. Here, the external force may be a force physically applied directly to the surgical robot or a force applied through the manipulation of the control device for the movement of the surgical robot, as described above. The user may further shift the surgical robot to the position of the virtual path point A2 so that the surgical robot is located in the moving path.

However, when the user shifts the surgical robot to the position of B2 and then stops the application of the external force, the body section 100 determine in what direction and by what distance the surgical robot departs from the moving path 810 with reference to the image information supplied from the camera unit 410 of the movement compensating device 400.

Referring to FIG. 13, the return path determining unit 1130 detects the position of a photographing area 1310 at which an area of interest 1320 is located with reference to the image supplied from the camera unit 410 and then creates and outputs a path return command for locating the center point of the area of interest 1320 at the center point of the photographing area 1310.

For example, when the moving path 810 is set in advance so as for the surgical robot to move centered on the operating table 150 (for example, in a circular orbit centered on the operating table) in the state where the center point of the area of interest 1320 is matched with the center point of the photographing area 1310, the return path determining unit 1130 can easily see whether the surgical robot is located in the predetermined moving path on the basis of only the positions of the center points of the area of interest 1320 and the photographing area 1310. The return path determining unit 1130 can recognize the presence and position of the area of interest 1320 by extracting an outline through the use of an image recognition technique. The return path determining unit 1130 can use analysis/comparison information of two or more recognition points to accurately analyze the movement and rotation.

The path return command may include information on the rotating direction and the rotation number of the multi-directional wheel 120. In this case, the information on the moving distance by which the multi-directional wheel 120 should rotate in practice with respect to the distance and the angle between the center point of the area of interest 1320 and the center point of the photographing area 1310 in the image information supplied from the camera unit 410 is stored in advance in the storage unit 720.

The return path determining unit 1130 may output a command for stopping the process of matching the screen center point 520 with the recognition points 510 and 540 to the movement compensating device 400 while an external force is being sensed, so that the information on the rotating direction and the rotation number included in the path return command becomes more accurate.

When the area of interest is not recognized in the photographing area 1310, the return path determining unit 1130 stores the direction (that is, the direction in which the area of interest 1320 moves from the center point of the photographing area 1310) in which the external force is first applied, first creates and outputs a path return command for causing the surgical robot to move in the opposite direction of the stored direction, and re-creates and outputs a path return command based on the above-mentioned method when the area of interest 1320 is visualized in the photographing area 1310.

When only a part of the area of interest 1320 is recognized in the photographing area 1310 and the center point of the area of interest 1320 is not recognized, the return path determining unit 1130 considers the center point of the currently-visualized part of the area of interest 1320 as the substantial center point of the area of interest 1320 until the substantial center point of the area of interest 1320 is recognized.

Hitherto, it has been stated that the surgical robot is returned to a predetermined moving path 810 and moves based on the position shift command when the surgical robot departs from the moving path 810 with an external and then it is recognized that an external force is not applied.

However, plural moving paths for the movement of the surgical robot may be formed in advance, for example, plural circular shapes having different radii. In this case, when the surgical robot departs from a first moving path with an external force and is located in a second moving path during the movement of the surgical robot along the first moving path and when it is recognized that the external force is not applied any more, the surgical robot is not returned to the first moving path but moves along the second moving path based on the position shift command.

For example, when the fluorescent dye drawn on the floor or ceiling of the operating room is recognized or the magnetic rail is sensed by a recognizer, the surgical robot can recognize that it is located in the moving path. When the fluorescent dye or the magnetic rail is not recognized, the surgical robot may move along the moving path recognized at the first time during the movement in the opposite direction of the direction in which the external force is applied as described above.

In this way, when the surgical robot departs from the existing moving path and moves along a moving path other than the existing moving path, the return path determining unit 1130 is referred to as a path resetting unit.

FIG. 14 is a flowchart illustrating the path returning method of the surgical robot according to still another exemplary embodiment of the invention.

Referring to FIG. 14, in step 1410, the body section 100 receives a position shift command from the master robot and stores the received position shift command in the storage unit 720. The position shift command includes at last destination position information.

In step 1420, the body section 100 determines whether an obstacle is present in the moving path 810 using the sensing signal output from the proximity sensor unit 1110.

When it is determined that an obstacle is not present, the process of 1460 is performed. When it is determined that an obstacle is present, the process of step 1430 is performed.

In step 1430, the body section 100 controls the operation of the multi-directional wheel 120 to stop the movement of the surgical robot. At this time, the alarm unit may give an alarm in a visual type or/and an auditory type.

In step 1440, the body section 100 determines whether the external force applied to the body section 100 is ended by the use of the sensing signal of the external force detecting unit 1120. Here, the external force may be a force physically applied directly to the surgical robot or a force applied by the manipulation of the control device for the movement of the surgical robot as described above.

When it is determined that the external force is continuously applied, the body section 100 waits in step 1440. In this case, the surgical robot is made to move in the direction of the external force by the magnitude of the external force.

However, when the applied external force is stopped, the body section 100 outputs a path return control signal for locating the center point of the area of interest 1320 at the center point (that is, the screen center point) of the photographing area 1310 to the multi-directional wheel 120 in step 1450.

Thereafter, the body section 100 having been returned to the predetermined moving path 810 outputs a control signal for the movement corresponding to the position shift command received in step 1410 to the multi-directional wheel 120 in step 1460.

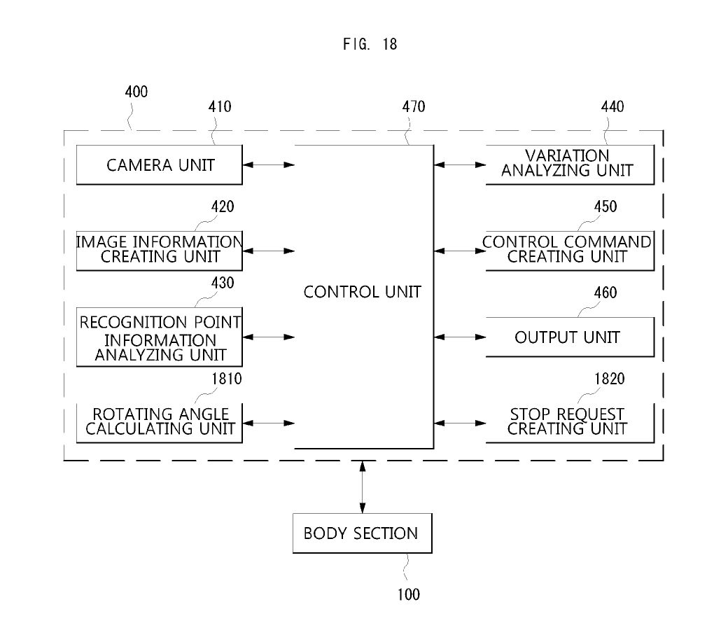

FIG. 15 is a diagram schematically illustrating the configuration of a master robot according to still another exemplary embodiment of the invention. FIG. 16 is a diagram illustrating an example of a screen display for the movement of the surgical robot according to still another exemplary embodiment of the invention.

As described above, a master robot 1500 may be incorporated into the surgical robot (that is, a slave robot) including the body section 100 or may be connected thereto via a communication network.

Referring to FIG. 15, the master robot 1500 includes a communication unit 1510, a display unit 1520, an input unit 1530, a movement information creating unit 1540, a posture information creating unit 1550, a command creating unit 1560, and a control unit 1570.

The communication unit 1510 is coupled to the body section 100 of the surgical robot via a wired or wireless communication network, transmits one or more of the position shift command and the surgical instrument manipulating command to the body section 100, and receives image information captured by one or more the camera unit 410 and the endoscope inserted into a human body from the body section.

The communication unit 1510 may further receive an image signal related to the situation of the operating room from a ceiling camera unit 1590 installed on the ceiling of the operating room via a wired or wireless communication network from the master robot 1500. The ceiling camera unit 1590 includes, for example, an image sensor.

The display unit 1520 outputs the image information received via the communication unit 1510 and captured by the camera unit 410 and/or the endoscope and the image information captured by the ceiling camera unit 1590 as visual information. A display example of the image information captured by the ceiling camera unit 1590 is shown in FIG. 16 and includes the information of the position of the operating table 150 and the position of the surgical robot as visual information. The image information captured by the ceiling camera unit 1590 may be displayed on the display unit 1520 as actual image information, or may be replaced with predetermined icons or figures through the use of the analysis of the image information and may be displayed on the display unit 1520.

The display unit 1520 may further display information related to the patient (such as a heart rate and a reference image (for example, a CT image and an MRI image)).

The display unit 1520 may be embodied to include one or more monitor devices. When the display unit 1520 is embodied by a touch screen, the display unit may further perform the function of the input unit 1530.

The input unit 1530 is a unit used to input the surgical instrument manipulating command the position shift command.

The input unit 1530 may include one or more control devices so as to input the surgical instrument manipulating command. The control device may be, for example, plural handles embodied to perform a surgical operation (such as the moving operation, the rotating operation, and the cutting operation of the robot arm by allowing an operator's hands to grasp and manipulate the handles. When the control device is embodied as a handle, the control device may include a main handle and a sub handle. An operator may manipulate the robot arm or the endoscope of the slave robot by the use of only the main handle or may manipulate the sub handle to operate plural surgical instruments at the same time. The main handle and the sub handle have various mechanical structures depending on the manipulation method thereof and may be embodied in various input units for operating the robot arm and/or other surgical instruments of the surgical robot, such as a joystick type, a keypad, a track ball, and a touch screen. The shape of the control device is not limited to the handle, but any shape may be employed as long as it can control the operation of the surgical robot via a wired or wireless communication network.

The input unit 1530 may further include an instruction unit inputting the position shift command to the surgical robot. The instruction unit may be embodied as a touch screen, a mouse used to point any position of the visual information displayed on the display unit 1520, a keyboard, and the like. The course of inputting a position shift command by the use of the input unit 1530 will be described in detail later with reference to the relevant drawings.

The movement information creating unit 1540 creates position shift information for causing the body section 100 to move to a position designated by an operator through the use of the input unit 1530 from the image information of the operating room captured by the ceiling camera unit 1590 and displayed on the display unit 1520.

The movement information creating unit 1540 may perform a conversion process of converting the distance and the angle between the points designated by the operator through the screen into the moving direction and the moving distance of the body section 100 used to actually move in creating the position shift information. The conversion reference information for the angle calculating method based on the reference direction and the method of converting the distance on the screen into the actual moving distance may be stored in the storage unit (not shown) in advance for the purpose of the conversion process.

The posture information creating unit 1550 creates posture information for causing a specific part (for example, the front surface) to face the operating table 150 or to face a side designated by a user at the time of the movement of the body section 100 corresponding to the position shift information created by the movement information creating unit 1540. The posture information for disposing the surgical robot with a posture suitable for performing the operation may be information for causing the body section 100 to rotate so as to direct the designated point to the front surface of the body section 100 when an operator designates the rotating angle and the rotating direction of the body section 100 at a fixed position through the use of the input unit 1530 or designates a point around the body section 100 in the image information of the operating room.

The command creating unit 1560 creates a position shift command corresponding to the position shift information created by the movement information creating unit 1540 and a posture control command corresponding to the posture information created by the posture information creating unit 1550, and transmits the created commands to the body section 100 via the wired or wireless communication network. The command creating unit 1560 further creates a surgical instrument manipulating command corresponding to the surgical instrument manipulating information input through the input unit 1540 from the operator and transmits the created command to the body section 100. The body section 100 is controlled in accordance with the position shift command, the posture control command, and/or the surgical instrument manipulating command supplied from the command creating unit 1560.

The control unit 1570 controls the operations of the constituent elements of the master robot 1500.

FIG. 16 shows the image information of the operating room captured by the ceiling camera unit 1590 and displayed on the display unit 1520 for the movement of the surgical robot.

The pixels of the image information of the operating room displayed on the display unit 1520 may be set in advance so that the positions thereof are specified as relative coordinates or absolute coordinates. When each pixel is specified as a relative coordinate, the leftmost and lower most point can be defined as (0, 0) as shown in the drawing and the coordinates of the pixels can be specified relative to the point.

In exemplarily describing the movement of the surgical robot with reference to FIG. 16, it is assumed that the current position of the body section 100 is a position P0 with a relative coordinate (50, 25), the destination position is a position P3 with a relative coordinate (48, 115), and the operating table 150 is interposed between the positions P0 and P3.

The operator sequentially designates the position P1 with a relative coordinate (10, 20) and the position P2 with a relative coordinate (10, 95) as the path points via which the body section 100 is made to move from the position P0 to the position P3 with reference to the image information of the operating room displayed on the display unit 1520. The position P3 may be designated after the position P2 is designated, and the position P0 may be designated before the position P1 is designated.

When the operator completes the designation of the positions through the use of the input unit 1530, the movement information creating unit 1540 recognizes the distance and the direction between the designated positions using the relative coordinates and creates the position shift information which is information of the rotating direction (that is, the moving direction of the body section 100) and the rotation number (that is, the moving distance of the body section 100) of the multi-directional wheel 120 with reference to the conversion reference information stored in advance in the storage unit.

For example, when the body section moves from the position P0 to the position P1, the movement information creating unit 1540 calculates the inclined angle and the distance using the relative coordinates and the triangular functions, and then creates the position shift information including the calculated angle (for example, −7 degrees) as the moving direction and including the moving distance (for example, 8 rotations) obtained by calculating the distance using the conversion reference information. When the angle is calculated on the basis of the predetermined reference line (for example, the horizontal straight line of the operating room and the reference line of the rotating direction of the multi-directional wheel 120 is set to the horizontal straight line of the body section 100, the lower shape of the body section 100 is recognized from the image information of the operating room through the use of the image recognition technique (such as an edge detection technique) and then the rotating direction may be re-calculated on the basis of the reference direction corresponding to the lower shape of the body section 100.

In this way, by sequentially creating the position shift information of the path points and the destination position designated by the operator and transmitting the position shift command corresponding thereto to the body section 100, the surgical robot (that is, the body section 100) can be made to move in the direction and to the position designated by the operator.

In this case, the surgical instruments and the like should be disposed to face the patient on the operating table 150 when the surgical robot moves to the designated position. This is intended for the safety of a patient or the like when the surgical robot moves in the state where the surgical instrument is inserted into the human body.

When the operator designates the operating table 150 to create the posture control command for the purpose of the posture control of the surgical robot before, during, or after the position selection for the movement of the body section 100, the surgical robot is controlled so that the multi-directional wheel 120 rotates in the state where the surgical operation processing unit 140 faces the patient, as shown in FIG. 16.

Hitherto, the method of controlling the movement of the surgical robot using the image information captured by the ceiling camera unit 1590 has been stated. However, the movement of the surgical robot may be controlled using an optical tracker, a magnetic tracker, and other position trackers without using the ceiling camera unit 1590, as described above.

The surgical robot has only to recognize the positional relation with the operating table 150 without installing the camera on the ceiling of the operating room. Accordingly, by attaching a recognition marker to the operating table 150 and mounting a camera on the surgical robot, a method of recognizing the positional relation therebetween and causing the surgical robot to move may also be employed.

FIG. 17 is a flowchart illustrating the movement processing method of the surgical robot according to still another exemplary embodiment of the invention.

Referring to FIG. 17, the master robot 1500 displays image information (that is, the image information of the operating room) obtained by processing an image signal supplied from the ceiling camera unit 1590 on the display unit 1520 in step 1710.

In step 1720, the master robot 1500 receives the path point position information and the destination position information input through the input unit 1530 from the operator with reference to the image information of the operating room displayed on the display unit 1520 for the purpose of the movement control of the surgical robot. At this time, the posture information for the posture control of the surgical robot, as described above.

In step 1730, the master robot 1500 creates a position shift command for causing the surgical robot to sequentially move to the positions with reference to the path point position information and the destination position information input in step 1720 and the conversion reference information stored in advance in the storage unit, and transmits the created position shift command to the body section 100 via the wired or wireless communication network. At this time, a posture control command for the posture control of the surgical robot may be further created and transmitted to the body section 100 via the wired or wireless communication network.

In response to the position shift command transmitted in step 1730, the body section 100 controls the multi-directional wheel 120 to move to the destination position designated by the operator.

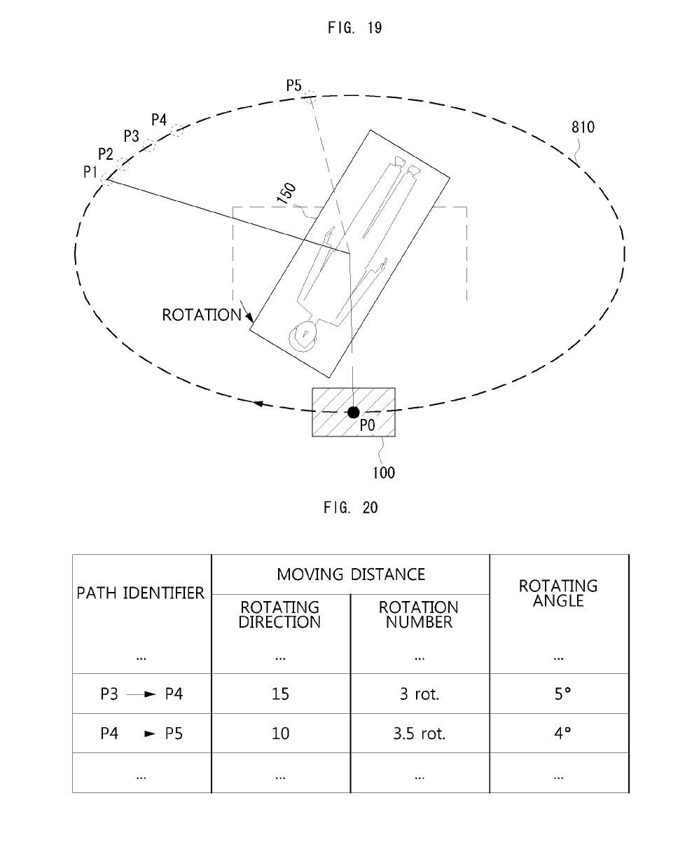

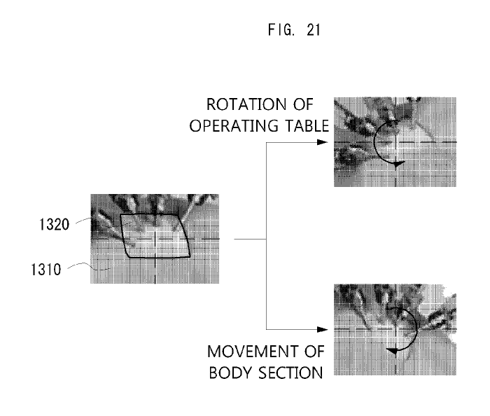

FIG. 18 is a block diagram illustrating the configuration of a movement compensating device according to still another exemplary embodiment of the invention. FIG. 19 is a conceptual diagram illustrating a movement compensating method of the movement compensating device according to still another exemplary embodiment of the invention. FIG. 20 is a diagram illustrating an example of control reference information of the multi-directional wheel according to still another exemplary embodiment of the invention. FIG. 21 is a diagram illustrating the concept of calculating a rotating angle according to still another exemplary embodiment of the invention.

Referring to FIG. 18, the movement compensating device 400 includes a camera unit 410, an image information creating unit 420, a recognition point information analyzing unit 430, a variation analyzing unit 440, a control command creating unit 450, an output unit 460, a rotating angle calculating unit 1810, a stop request creating unit 1820, and a control unit 470. As described above, the movement compensating device 400 may be disposed in the body section 100 or the surgical operation processing unit 140 and supplies a control command for causing the surgical operation processing unit 140 to move to the coupling unit 130.

The camera unit 410 outputs an image signal created by capturing an image of an operating site. The camera unit 410 includes, for example, an image sensor.

The image information creating unit 420 processes the image signal input from the camera unit 410 and creates image information to be displayed on a display device (not shown) installed in or coupled to the master robot. The image information to be created by the image information creating unit 420 can be created in the image format in which pixel information can be analyzed by the recognition point information analyzing unit 430.

The recognition point information analyzing unit 430 creates coordinate information of an object included in the image information created by the image information creating unit 420 and analysis information of the distance and the angle relative to a reference point. The object analyzed by the recognition point information analyzing unit 430 may be the recognition marker 350 formed at one end of the upper trocar housing 310 of the medical trocar 300 described above with reference to FIG. 3, a specific site (for example, a navel), a specific site of an operating cover.

The variation analyzing unit 440 creates variation information in the distance and the angle between the analysis information pieces created to correspond to the image frames by the recognition point information analyzing unit 430.

The control command creating unit 450 creates a control command for controlling the coupling unit 130 so that the variation information created by the variation analyzing unit 440 becomes 0 (zero). The control command serves to cause the body section to move in the translational and/or rotational moving manner in the direction and by the distance by which the position of the recognition point is fixedly maintained with the movement of the coupling unit 130 (that is, by which the variation information of the surgical operation processing unit 140 is 0). The position of the surgical operation processing unit 140 can be kept at the position before the body section 100 moves by the adjustment of the coupling unit 130 corresponding to the control command, even when the body section 100 moves in any direction.

The output unit 460 outputs the control command created by the control command creating unit 450 to the coupling unit 130 so as to keep the image input from the camera unit 410 constant (that is, so as to keep the position of the surgical operation processing unit 140 relative to the patient on the operating table 150 constant within a margin of error).

When the operating table 150 is recognized to rotate by calculating the rotating angle of the rotating angle calculating unit 1810, the output unit 460 also outputs stop request information created by the stop request creating unit 1820 to the body section 100.

The output unit 460 may transmit the control command to the master robot so as to recognize the state of the coupling unit 130 for keeping the position of the surgical operation processing unit 140 or may transmit the image information created by the image information creating unit 420 to the master robot so as to display the image information on the display device (not shown) installed in or coupled to the master robot.

The rotating angle calculating unit 1810 creates rotating angle information indicating by what degrees the surgical robot or/and the operating table 150 rotates about a center point using the image information created by processing the image signal input from the camera unit 410 and the control reference information stored in advance in the storage unit (not shown). Here, the center point may be, for example, a vertical and horizontal center point of the operating table 150 or a center point of the operating site.

The rotating angle calculating unit 1810 can create the information on by what degrees the operating table 150 rotates using the variation information in the angle analyzed by the variation analyzing unit 440 and the created rotating angle information can be supplied to the body section 100. The rotating angle calculating unit 1810 can recognize the remainder rotating angle in moving to the destination position corresponding to the position shift command received from the master robot and can transmit the rotating angle information in the respective analysis steps and/or the calculated remainder rotating angle information to the body section 100 for use in control of the multi-directional wheel 120.

The stop request creating unit 1820 creates stop request information for stopping the movement of the body section 100 in response to the position shift command and outputs the created stop request information to the body section 100 via the output unit 460, when it is determined by the rotating angle calculating unit 1810 that the remainder rotating angle is 0 (zero). When a constituent element (for example, the wheel manipulating unit 740) of the body section 100 determines whether the remainder rotating angle is 0 on the basis of the rotating angle information supplied from the rotating angle calculating unit 1810, the stop request creating unit 1820 may be made unnecessary.

The control unit 470 controls the constituent elements of the movement compensating device 400 to perform the above-mentioned functions.

FIG. 19 conceptually shows the movement compensating method of the movement compensating device, FIG. 20 shows the control reference information of the multi-directional wheel 120, and FIG. 21 shows the concept of calculating the rotating angle.

As shown in FIG. 19, for the purpose of the smooth surgical operation, the surgical robot may be made to move along a predetermined moving path 810 or the operating table 150 may be made to rotate. Here, the moving path 810 may include plural virtual path points and the virtual path points may be arranged continuously or discretely.

When the surgical robot is made to move from the current position along the moving path 810, the rotating angle from the current position to the destination position can be used. For example, when it is instructed to move from the position P0 as the current position to the position P5, the rotating angle calculating unit 1810 and/or the body section 100 can recognize that the position shift command indicates the rotation about a center point along the predetermined moving path 810 by 170 degrees.

In response to the position shift command, the body section 100 controls the multi-directional wheel 120 to move to the destination position via the virtual path points with reference to the control reference information shown in FIG. 20. The control reference information includes information on the rotating angle about the center point in the movement between the virtual path points, and the body section 100 can recognize whether it rotates by the angle corresponding to the target rotating angle information (that is, the rotating angle information from the current position to the destination position).

When the target rotating angle information corresponding to the position shift command from the master robot 1500 or the target rotating angle information corresponding to the position shift command from the body section is supplied, the rotating angle calculating unit 1810 can recognize by what degree it should rotationally move about the center point along the predetermined moving path 810, and can check whether the remainder rotating angle information (that is, the value obtained by subtracting the rotating angle information corresponding to the variation information from the target rotating angle information) is 0 (zero) with reference to the variation information of the angle supplied from the variation analyzing unit 440. When the body section 100 is configured to continuously move until the stop request information is received from the movement compensating device 400, the rotating angle calculating unit 1810 may control the stop request creating unit 1820 so as not to create the stop request information until the remainder rotating angle information becomes 0.

However, when the operator designates the body section located at the position P0 to move to the position P5 and the operating table 150 is additionally made to rotate during the movement of the surgical robot, to what position the body section 100 should move may be a problem. This is because the initially-designated position P5 is the best position suitable for performing the subsequent surgical operation on the patient on the operating table 150.

Therefore, when the operating table 150 rotates in a certain direction by a certain angle, the position P5 which is the initially-designated destination position should be changed to the position P1 so as to correspond to the rotation of the operating table 150. In order to accurately determine the changed destination position, the surgical robot needs to stop the position shift until the rotation of the operating table 150 is ended, when the rotation of the operating table 150 is recognized.

That is, while the body section 100 is moving to the destination position via the virtual path points on the basis of the control reference information, the body section is supplied with the rotating angle information based on the variation information in the angle analyzed by the variation analyzing unit 440 from the rotating angle calculating unit 1810 and determines whether the supplied rotating angle information is matched with the rotating angle information included in the control reference information within a margin of error. When both rotating angle information pieces are not matched within the margin of error, it is recognized that the operating table 150 rotates and the operation of the multi-directional wheel 120 is stopped to stop the movement of the surgical robot. When the rotating angle information other than 0 (zero) is received from the rotating angle calculating unit 1810 after the movement of the surgical robot is stopped, it means that the rotation of the operating table 150 is kept and thus the rotating angle of the operating table 150 should be reflected in the remainder rotating angle information so as to allow the surgical robot to move to an appropriate position.

It is assumed that the operating table 150 rotates in the direction (that is, the opposite direction of the rotating direction of the surgical robot) of the arrow shown in FIG. 19 while the surgical robot is rotating along the moving path 810 indicated by the direction of the arrow shown in FIG. 19. Then, the image information (see (a) of FIG. 21) created by the image information creating unit 420 is displayed to rotate in the directions (see (b) and (c) of FIG. 21).

The image information displayed to rotate in the directions is controlled so that the recognition point is located at the screen center point as described with reference to FIG. 4B or the like through the processes of the variation analyzing unit 440 and the control command creating unit 450, and it can be recognized through the control by what degree in what direction the image information rotates.

As shown in FIG. 21, when the rotating direction of the operating table 150 is opposite to the rotational moving direction of the surgical robot, the remainder rotating angle information (that is, the destination position information) can be updated by subtracting the rotating angle of the operating table 150 from the remainder rotating angle information. However, when the rotating direction of the operating table 150 is equal to the rotational moving direction of surgical robot, the destination position information can be updated by adding the rotating angle of the operating table 150 to the remainder rotating angle information.

The body section 100 recognizes the rotating angle information supplied from the rotating angle calculating unit 1810 in the state where the movement is stopped as the rotating angle information based on the rotation of the operating table 150 and updates the remainder rotating angle information. The updated remainder rotating angle information can be supplied again to the movement compensating device 400, and the surgical robot will move along the predetermined moving path 810 until the updated remainder rotating angle information becomes 0.

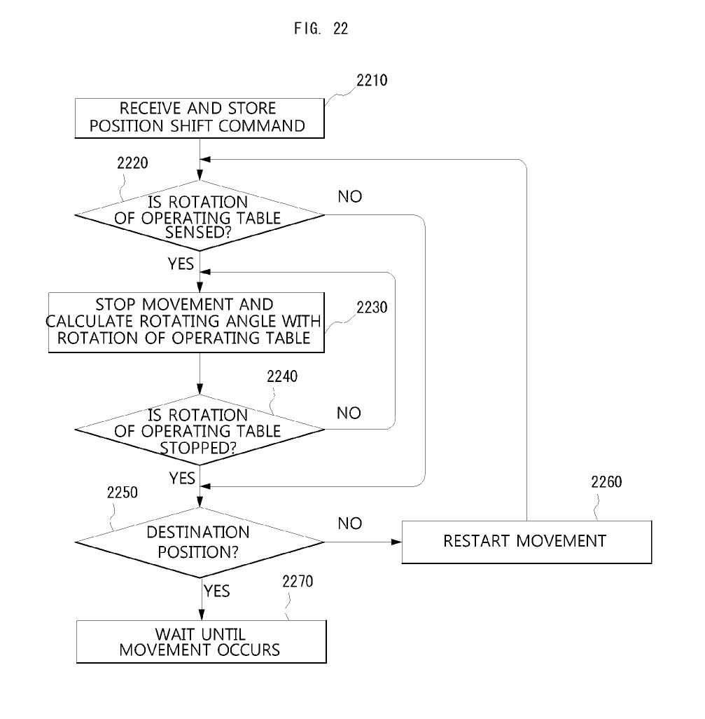

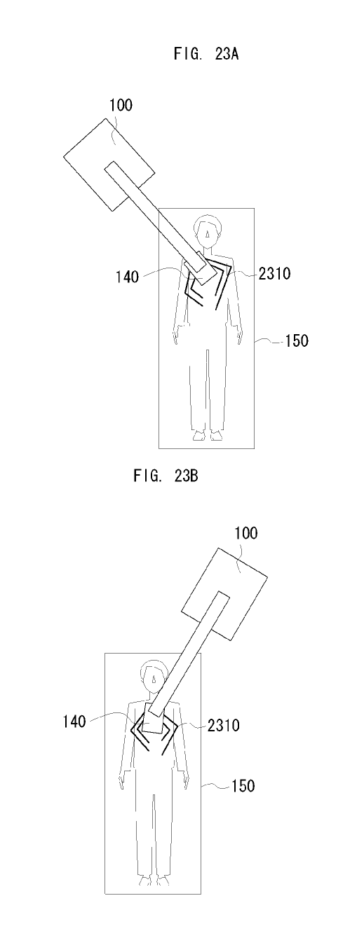

FIG. 22 is a flowchart illustrating a movement processing method of the surgical robot according to still another exemplary embodiment of the invention.

Referring to FIG. 22, the body section 100 receives and stores a position shift command or/and a target rotating angle information (that is, the rotating angle information from the current position to the destination position) transmitted from the master robot 1500 in step 2210.

In step 2220, the body section 100 determines whether the operating table 150 rotates on the basis of the rotating angle information supplied from the movement compensating device 400 and created by analyzing and calculating the image information corresponding to the image signal supplied from the camera unit 410. The body section 100 can recognize that the operating table 150 rotates, when a rotating angle greater or smaller by the margin of error than the rotating angle (see FIG. 20) predicted with the rotational movement of the surgical robot based on the position shift command is recognized and supplied through the image information analysis.

When it is recognized that the operating table 150 rotates, the process of step 2230 is performed. Otherwise, the process of step 2250 is performed.

In step 2230, the body section 100 accurately calculates the rotating angle of the operating table 150, stops the movement of the multi-directional wheel 120 for the purpose of correcting the destination position, and calculates the rotating angle of the operating table 150 with reference to the rotating angle information supplied from the movement compensating device 400. The movement compensating device 400 can analyze the image information corresponding to the image signal supplied from the camera unit 410 to calculate the rotating angle corresponding to the rotation of the operating table 150 on the basis of the variation information in the angle between the analysis information created by the variation analyzing unit 440. The body section 100 can reflect the rotating angle information corresponding to the rotation of the operating table 150 to update the remainder rotating angle information.

In step 2240, the body section 100 determines whether the rotation of the operating table 150 is stopped using the rotating angle information supplied from the movement compensating device 400.

When it is determined that the rotation of the operating table 150 is not stopped, the process of step 2230 is performed again. When it is determined that the rotation of the operating body 150 is stopped, the process of step 2250 is performed.

In step 2250, the body section 100 determines whether the remainder rotating angle information is 0 (that is, whether the current position of the surgical robot is the destination position based on the position shift command).

When it is determined that the current position is not the destination position, the body section 100 restarts the movement to the destination position in step 2260 and then the process of step 2220 is performed again.

However, when it is determined in step 2250 that the current position is the destination position, the body section 400 waits until a subsequent command (for example, a surgical instrument manipulating command and a position shift command) is received in step 2270.

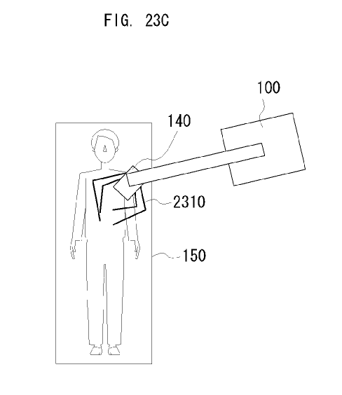

FIGS. 23A to 23C are conceptual diagrams illustrating the movement of a surgical robot according to still another exemplary embodiment of the invention.

That is, FIGS. 23A to 23C are diagrams illustrating the relationship between a patient and the body section 100, the surgical operation processing unit 140, and the operating table 150 before and after the body section 100 moves. For the purpose of simplification of the drawings, the instrument and the like included in the surgical operation processing unit 140 are not shown in the drawings.

As shown in FIGS. 23A to 23C, when the body section 100 is intended to move from the right side of the patient's head to the left side, the body section 100 sequentially moves to the positions shown in FIGS. 23B and 23C by controlling the operation of the multi-directional wheel 120.

However, the surgical operation processing unit 140 shown in FIGS. 23B and 23C is controlled so that the position and the direction relative to the patient are not fixed, unlike the above description.

That is, when the operator wants to display image information which is not identical to the image input at the position shown in FIG. 23A during the movement of the body section 100 or intentionally wants to display different image information by controlling the position of the surgical operation processing unit 140, it is possible to control the position and the direction of the surgical operation processing unit 140 by appropriately controlling the coupling unit 130. In this case, the body section 100 needs to control the position of the robot arm and the insertion position of the instrument 2310 so that an excessive force is not applied to the insertion position so as for the instrument or the like inserted into the human body not to damage the patient's skin, organism, and the like.

That is, when it is not necessary to always match the screen desired by a user with the initial screen, it is possible to display image information desired by the user, by appropriately controlling the position and/or the direction of the surgical operation processing unit 140 in consideration of the relative position to the operating table 150. The control method of the coupling unit 130 for this purpose can be obvious from the technical concept described in this specification and thus description thereof will not be made.

When an operator wants to display identical image information during the movement of the body section 100, the position and the direction of the surgical operation processing unit 140 can be made to be fixed with respect to the patient by controlling the coupling unit 130 as described above.

The above-mentioned movement controlling/compensating method of a surgical robot using a camera image can be embodied as a time-series automated procedure by a software program built in a digital processor or the like. Codes and code segments of the program will be easily thought out by computer programmers skilled in the art. The program can be stored in a computer-readable recording medium and can be read and executed by a computer so as to embody the above-mentioned method. Ex amples of the recording medium include a magnetic recording medium, an optical recording medium, and a carrier wave medium.

While the invention is described with reference to the exemplary embodiments, it will be understood by those skilled in the art that the invention can be modified and changed in various forms without departing from the concept and scope of the invention described in the appended claims.

What is claimed is:

1. A movement compensating device of a surgical robot in which a surgical operation processing unit mounted with a surgical instrument is coupled to one end of a body section, comprising:
an image information creating unit that creates image information corresponding to an image signal supplied from a camera unit having captured an image of an operating site;
a recognition point information analyzing unit that creates analysis information on a distance and an angle between a recognition point recognized from image information pieces corresponding to a predetermined number of image frames and a predetermined reference point;
a variation analyzing unit that creates variation information in the distance and the angle between two analysis information pieces continuously created; and
a control command creating and outputting unit that creates and outputs a control command for adjusting the position of the surgical operation processing unit so that the variation in distance and angle included in the variation information be 0 (zero).

2. The movement compensating device according to claim 1, wherein the camera unit is disposed at one end of the surgical operation processing unit.

3. The movement compensating device according to claim 1, wherein a movement unit that allows the body section to move in any direction is disposed under the body section.

4. The movement compensating device according to claim 3, wherein the movement unit includes an omnidirectional wheel.

5. The movement compensating device according to claim 3, wherein the movement unit is embodied in the form of one or more of a magnetic levitation type and a ball wheel type.

6. The movement compensating device according to claim 1, wherein each recognition point is an object which is included in the image frames so as to be recognized as an object by capturing an image of a recognition marker formed at one end of a medical trocar or a predetermined feature point to be included in the image information.

7. The movement compensating device according to claim 1, wherein the surgical operation processing unit and one end of the body section are coupled to each other through the use of a coupling unit, and
wherein the coupling unit includes a motor assembly that is adjusted to allow the surgical operation processing unit to rotate and to move in a horizontal direction in response to the control command.

8. A movement compensating method of a surgical robot, which is performed by a movement compensating device, comprising:
creating image information corresponding to an image signal supplied from a camera unit having captured an image of an operating site;
creating analysis information on a distance and an angle between a recognition point recognized from image information pieces corresponding to a predetermined number of image frames and a predetermined reference point;
creating variation information of the distance and the angle between two analysis information pieces continuously created; and
creating and outputting a control command for adjusting the position of a surgical operation processing unit so that the variation in distance and angle included in the variation information be 0 (zero).

9. The movement compensating method according to claim 8, wherein the surgical robot includes a body section and the surgical operation processing unit that is mounted with a surgical instrument and that is coupled to one end of the body section, and
wherein the camera unit is disposed at one end of the surgical operation processing unit.

10. The movement compensating method according to claim 9, wherein a movement unit that allows the body section to move in any direction is disposed under the body section.

11. The movement compensating method according to claim 10, wherein the movement unit includes an omnidirectional wheel.

12. The movement compensating method according to claim 10, wherein the movement unit is embodied in the form of one or more of a magnetic levitation type and a ball wheel type.

13. The movement compensating method according to claim 8, wherein each recognition point is an object which is included in the image frames so as to be recognized as an object by capturing an image of a recognition marker formed at one end of a medical trocar or a predetermined feature point to be included in the image information.

14. The movement compensating method according to claim 9, wherein the surgical operation processing unit and one end of the body section are coupled to each other through the use of a coupling unit, and
wherein the coupling unit includes a motor assembly that is adjusted to allow the surgical operation processing unit to rotate and to move in a horizontal direction in response to the control command.

15. A surgical robot comprising:
a movement unit that enables the surgical robot to move in any direction;
a communication unit that receives a position shift command for causing the movement unit to move; and
a movement processing unit that creates a control signal for causing the movement unit to move along a predetermined moving path in response to the position shift command.

16. The surgical robot according to claim 15, further comprising a storage unit that stores movement information on a moving direction and a moving distance of the movement unit so as to correspond to the position shift command,
wherein the control signal is a signal for causing the movement unit to move on the basis of the movement information corresponding to the position shift command.

17. The surgical robot according to claim 16, wherein the movement information includes information on the moving directions and the moving distances of movements between a plurality of virtual path points included in the predetermined moving path.

18. The surgical robot according to claim 17, wherein the predetermined moving path is drawn with a fluorescent dye on the floor or ceiling of an operating room so as to be recognized by a recognizer of the surgical robot and to move along the recognized moving path or is formed in the form of a magnet or magnetic rail under the floor of the operating room so as to induce the surgical robot to move.

19. The surgical robot according to claim 17, further comprising a sensor that senses the presence of an object coming near and that outputs a sensing signal,
wherein the movement processing unit outputs a stop command for stopping the movement of the movement unit to the movement unit or stops creating and outputting the control signal for causing the movement unit to move, when the sensing signal is output from the sensor.

20. The surgical robot according to claim 15, wherein the movement unit includes an omnidirectional wheel.

21. The surgical robot according to claim 15, wherein the movement unit is embodied in the form of one or more of a magnetic levitation type and a ball wheel type.
